SEQUENCE LISTING (16 sequences)

SEQ ID NO: 1 - DNA, 2233 bp, Bacillus subtilis
atggtcagcatccgccgcagcttcgaagcgtatgtcgatgacatgaatatcattactgttctgattcctgctgaacaaaaggaaatcatgacaccgccgtttcggcttgagacagaaataacagattttcctctggctgtcagggaggaatactcccttgaagcaaaatacaagtacgtctgcgtatccgaccatcctgtgacatttggaaaaatccattgcgtcagagcatccagcggccacaaaacggatctccaaattggcgcggtcatccggacggcagcgtttgatgacgaattttattatgacggagagctgggcgccgtttataccgcggatcataccgtatttaaagtatgggcgcctgctgcaacctcagctgctgtcaagctttcacaccccaataaaagcgggcgcacattccaaatgactcgcttggaaaaaggcgtctatgccgttacggtcacaggtgaccttcacggatatgagtatttgttttgcatctgcaacaattcagaatggatggaaacagttgaccagtatgccaaggctgtgactgtaaatggagagaagggcgtcgtcttgcgcccggatcaaatgaaatggactgctcctcttaaaccattctcacaccctgtggatgccgtcatctatgagacgcatcttcgcgacttctccatccatgaaaacagcggcatgataaacaagggaaaatacttagcgctgacggaaactgatacacaaaccgcaaatggcagttcttcgggattagcgtatgtaaaagagcttggtgtgacacatgtggagcttctgccggtgaatgattttgccggagttgatgaagagaagccgcttgatgcatacaattggggatataacccgcttcatttctttgccccggagggaagctatgcctcaaatcctcatgatcctcaaacgagaaaaacagagctgaaacaaatgatcaataccctgcatcagcacggtctgcgagtcattctggatgttgtttttaaccatgtgtataagagggagaattccccctttgaaaagacagtgcccggttattttttccggcacgacgaatgtgggatgccatcaaacggcaccggcgttggcaatgatattgcatcagaaagaaggatggcaagaaaattcattgcggattgcgtggtctattggcttgaagaatacaatgttgacggcttccgctttgatctcctcgggattttagatattgacaccgtgctttatatgaaagagaaagcaactaaggcaaagcccggaatcctgctttttggagaagggtgggacctggctacaccgctgccgcatgaacagaaagctgctttggcgaacgcgccaagaatgccgggcatcggcttttttaatgatatgtttcgtgacgctgtaaaagggaacacctttcaccttaaggcaacagggtttgcgctcggcaacggtgaatcagcacaagctgtgatgcatggaattgccgggtcttccggatggaaggcattagcaccgattgttccggaaccaagccagtccatcaattatgtcgaatcacacgacaatcacaccttttgggataaaatgagctttgcgcttcctcaagaaaatgacagccgaaagcgaagcaggcaaaggcttgcagtcgcgattattttgcttgcccaaggggtgccgtttattcacagcggccaggaatttttccggacgaagcagggagtggaaaacagctatcaatccagtgacagcatcaaccagctcgactgggatcgccgtgaaacattcaaagaagatgttcactatatccgcaggctgatctcgctgagaaaagcgcatcctgcattccgtcttaggtccgctgcagacatccagcgccatcttgaatgcttgacgctaaaagaacaccttatcgcatacaggctttatgatcttgacgaggttgacgaatggaaagatatcattgttatccatcacgcgagtccagactccgtcgagtggaggctgccaaacgacataccttatcggcttttatgtgatccatcaggatttcaggaagacccaacagaaatcaagaaaacggttgcagtaaacggcatcggaacggttatcttatatttagcatcagatcttaagagttttgcttgacgaagtttgtgctatagcgctaaaatttaaaggacggctattgattagcatatgagactctcaatagctgtctttt

SEQ ID NO: 2 - protein, 718 aa, Bacillus subtilis
MVSIRRSFEAYVDDMNIITVLIPAEQKEIMTPPFRLETEITDFPLAVREEYSLEAKYKYVCVSDHPVTFGKIHCVRASSGHKTDLQIGAVIRTAAFDDEFYYDGELGAVYTADHTVFKVWAPAATSAAVKLSHPNKSGRTFQMTRLEKGVYAVTVTGDLHGYEYLFCICNNSEWMETVDQYAKAVTVNGEKGVVLRPDQMKWTAPLKPFSHPVDAVIYETHLRDFSIHENSGMINKGKYLALTETDTQTANGSSSGLAYVKELGVTHVELLPVNDFAGVDEEKPLDAYNWGYNPLHFFAPEGSYASNPHDPQTRKTELKQMINTLHQHGLRVILDVVFNHVYKRENSPFEKTVPGYFFRHDECGMPSNGTGVGNDIASERRMARKFIADCVVYWLEEYNVDGFRFDLLGILDIDTVLYMKEKATKAKPGILLFGEGWDLATPLPHEQKAALANAPRMPGIGFFNDMFRDAVKGNTFHLKATGFALGNGESAQAVMHGIAGSSGWKALAPIVPEPSQSINYVESHDNHTFWDKMSFALPQENDSRKRSRQRLAVAIILLAQGVPFIHSGQEFFRTKQGVENSYQSSDSINQLDWDRRETFKEDVHYIRRLISLRKAHPAFRLRSAADIQRHLECLTLKEHLIAYRLYDLDEVDEWKDIIVIHHASPDSVEWRLPNDIPYRLLCDPSGFQEDPTEIKKTVAVNGIGTVILYLASDLKSFA

SEQ ID NO: 3 - DNA, 30 bp, Artificial
  PCR primer
ggccatggtcagcatccgccgcagcttcga

SEQ ID NO: 4 - DNA, 30 bp, Artificial
  PCR primer
ggctcgagtcaagcaaaactcttaagatct

SEQ ID NO: 5 - DNA, 33 bp, Artificial
  PCR primer
gtaaaagggaacaccggtcaccttaaggcaata

SEQ ID NO: 6 - DNA, 33 bp, Artificial
  PCR primer
gtaaaagggaacacctctcaccttaaggcaata

SEQ ID NO: 7 - DNA, 20 bp, Artificial
  PCR primer
ttattgccggagagtggcga

SEQ ID NO: 8 - DNA, 20 bp, Artificial
  PCR primer
ccagactgctgacaaagtgc

SEQ ID NO: 9 - DNA, 34 bp, Artificial
  PCR primer
ggaattccatatggatgtcgtcgtccgcttaccg

SEQ ID NO: 10 - DNA, 29 bp, Artificial
  PCR primer
gctctagattatttactgctcaccggcag

SEQ ID NO: 11 - DNA, 19 bp, Artificial
  PCR primer
gccggccgactccggtgac

SEQ ID NO: 12 - DNA, 3664 bp, Klebsiella pneumoniae
tggcgataagcgtggcgaatcataatcagtttgcggtttcgggtcgtcattccctttattgatttactcccccggcttcctacgccccccgctcatttgtgggggatgattgcgcctgggaaagcaaaaatatctaattagcgcgctgtaaagatattcatcctttcttaccttttataatttaataagcatattagggactatcgaatgctcagatatacctgtcatgccctatttcttggatcgttagtattattgagtggctgtgataacagctcttcctcttctacctctggctcaccgggttcaccaggcaatcctggcaacccaggcactcccggcacgcccgacccgcaggatgtcgtcgtccgcttaccggacgttgccgtcccaggcgaagcggtgcaggcttccgccaggcaggctgtcattcatctcgtcgatatcgccggcatcaccagcagcacgccggccgactatgcgacgaaaaacctctatttatggaacaacgaaacctgtgacgcgctgagcgcgccggtggcggactggaatgatgtcagcaccaccccgaccggcagcgacaaatatggcccttattgggtgatcccgctgactaaagagagcggatgcatcaacgttatcgtccgcgatggcaccaataagcttatcgacagcgacctgcgcgtctctttcagtgatttcaccgatcggacggtatcggtcatcgccggcaacagcgcggtctatgactcccgcgccgacgccttccgcgccgcctttggcgtggcgctggccgatgcgcactgggtcgataagactactctgctgtggccgggtggggaaaataaacccattgtgcgcctctattacagccacagcagtaaggtggccgccgacagtaacggcgaatttagcgataaatatgtcaagctgacccccaccaccgtcagccagcaggtaagcatgcgcttcccgcatctcgccagctatccagcctttaaactgccggatgatgttaacgtcgatgaattgctgcagggcgagacggtggcgatagccgcggaaagcgatgggatcctgagctcagccacccaggtgcagaccgccggcgtgctggacgatacctatgccgccgccgccgaggcgctgagctatggcgcccagctaaccgatagcggcgtgaccttccgcgtctgggcgcccacggcgcagcaggtcgagctggtgatctatagcgcggacaagaaagtgatagccagtcacccgatgacccgcgatagcgcctccggcgcctggtcctggcaggggggaagcgacctgaagggcgccttctaccgctacgcgatgacggtctaccacccgcagtcgcgtaaagtcgagcagtacgaagtgaccgatccctacgcccacagtttgtcgaccaactcggagtacagccaggtggtcgatctcaacgacagcgcgctgaagccggaaggctgggacgggctgacgatgccgcacgcgcagaaaaccaaagccgatctggcgaaaatgacgattcacgaatcgcatattcgcgatctctccgcctgggatcaaaccgtgcctgcggagctgcgcggtaagtatctggcgctcaccgcccaggaaagcaatatggtccagcatctgaaacagctgtcggcctcgggcgtgacccatattgagctgctgccggtcttcgatctggcgacggtcaatgagttcagcgacaaggtcgccgatattcagcagccgttcagccgcctgtgcgaggtcaatagcgcggtgaagagcagcgagttcgcgggctattgcgacagcggttcgacggtcgaagaggtgctgacccagctgaagcagaacgacagcaaggataacccgcaggtgcaggcgttgaatacgctggtggcgcagaccgactcctataactggggctacgatccgttccactacacggtaccggaaggatcctacgccaccgatccggaaggcacggcgcgtattaaagagttccgcaccatgattcaggcgatcaagcaggatctgggaatgaacgtcattatggacgtggtgtacaaccacaccaacgccgccggcccgaccgatcgcacctcggtactggataagatcgtcccctggtactaccagcgtctgaacgaaaccaccggcagcgtggaatcggctacctgttgctccgactcggcgccagagcaccggatgttcgccaagcttatcgccgattcactggcggtatggaccaccgattataagatcgatggcttccgcttcgacctgatgggctaccacccgaaagcgcagatcctctcggcctgggaacgcattaaagcgctgaacccggatatctacttctttggcgaaggctgggattccaaccagagcgatcgctttgaaattgcctcgcaaatcaatctcaaaggcaccgggatcggcacgttctccgatcgtctgcgcgacgccgtgcgcggcggcgggccgttcgactccggtgacgcattacgccagaaccagggcgtgggcagcggggctggcgttctgccgaatgagctgaccaccctgagcgacgatcaggcgcgtcacctcgccgatctgacccgtctcggcatggccggtaaccttgcggactttgtgctgatcgacaaagacggcgcggtgaagagaggcagcgagattgattataacggcgcgccaggcggctatgcggctgatccgacggaagtcgtgaactatgtgtcaaaacacgataaccagacgctgtgggacatgatcagctataaagccgctcaggaggcggatctcgatacccgcgtccggatgcaggcggtgtcgctggcgacggtgatgctcggccaagggatcgcctttgaccagcagggctcggagctgctgcgctctaaatcctttacccgcgattcgtatgattccggcgactggtttaaccgcgtggactactccctgcaggacaacaactacaacgtcggtatgccgcgcagcagcgatgatggcagcaattatgacattatcgcccgggtgaaagacgcggtggctactccgggtgaaacggagctcaagcagatgaccgcgttttatcaggagctgaccgcgctgcgtaaatcgtctccgctgtttaccctcggcgacggcgcgacggtgatgaagcgcgtggacttccgcaatactggcgccgatcagcagacgggtctgctggtgatgaccatcgatgacgggatgcaggctggcgccagtctggacagccgtgtcgacggcatcgtggtggcgatcaacgccgcgccggaaagccggacgctgcaggacttcgccggcacatcgctccagctgagcgctattcagcaggcggcgggcgaccggtcgctggcgagcggcgtgcaggttgccgctgacggttcggtcacgctgccggcctggtcggttgccgttctcgagttgccgcagggcgagtcgcagggcgctggcctgccggtgagcagtaaataacataacgtagggcgcccgtaagggcgcctctggctgatgacaaacctgtttcgttattgtgctcgcctttctgtcgggtggcggctttcgcttacccggcctacgaaagattgcaatttcaaacagttatgcagttatctgtaggccc

SEQ ID NO: 13 - protein, 1102 aa, Klebsiella pneumoniae
MLRYTCHALFLGSLVLLSGCDNSSSSSTSGSPGSPGNPGNPGTPGTPDPQDVVVRLPDVAVPGEAVQASARQAVIHLVDIAGITSSTPADYATKNLYLWNNETCDALSAPVADWNDVSTTPTGSDKYGPYWVIPLTKESGCINVIVRDGTNKLIDSDLRVSFSDFTDRTVSVIAGNSAVYDSRADAFRAAFGVALADAHWVDKTTLLWPGGENKPIVRLYYSHSSKVAADSNGEFSDKYVKLTPTTVSQQVSMRFPHLASYPAFKLPDDVNVDELLQGETVAIAAESDGILSSATQVQTAGVLDDTYAAAAEALSYGAQLTDSGVTFRVWAPTAQQVELVIYSADKKVIASHPMTRDSASGAWSWQGGSDLKGAFYRYAMTVYHPQSRKVEQYEVTDPYAHSLSTNSEYSQVVDLNDSALKPEGWDGLTMPHAQKTKADLAKMTIHESHIRDLSAWDQTVPAELRGKYLALTAQESNMVQHLKQLSASGVTHIELLPVFDLATVNEFSDKVADIQQPFSRLCEVNSAVKSSEFAGYCDSGSTVEEVLTQLKQNDSKDNPQVQALNTLVAQTDSYNWGYDPFHYTVPEGSYATDPEGTARIKEFRTMIQAIKQDLGMNVIMDVVYNHTNAAGPTDRTSVLDKIVPWYYQRLNETTGSVESATCCSDSAPEHRMFAKLIADSLAVWTTDYKIDGFRFDLMGYHPKAQILSAWERIKALNPDIYFFGEGWDSNQSDRFEIASQINLKGTGIGTFSDRLRDAVRGGGPFDSGDALRQNQGVGSGAGVLPNELTTLSDDQARHLADLTRLGMAGNLADFVLIDKDGAVKRGSEIDYNGAPGGYAADPTEVVNYVSKHDNQTLWDMISYKAAQEADLDTRVRMQAVSLATVMLGQGIAFDQQGSELLRSKSFTRDSYDSGDWFNRVDYSLQDNNYNVGMPRSSDDGSNYDIIARVKDAVATPGETELKQMTAFYQELTALRKSSPLFTLGDGATVMKRVDFRNTGADQQTGLLVMTIDDGMQAGASLDSRVDGIVVAINAAPESRTLQDFAGTSLQLSAIQQAAGDRSLASGVQVAADGSVTLPAWSVAVLELPQGESQGAGLPVSSK

SEQ ID NO: 14 - protein, 922 aa, Bacillus sp.
DGTTTNVIVHYFRPGGDYQSWSLWMWPEGGDGNNYNFNGTDSYGEIANVSIPGSPSKVGIIVRTQDWAKDVSQDRYIDLSKGHEVWLVQGNSQIFYNEKDAEDAAEPAVSNAYLDAPNKVLVKLSQPFTLGEGASGFTVHDDTANGDIPVTKVKNANKVEDVTAILAGTFQHIFGGSDWAPDNHSTQLKKVNDNLYQFSGELPGGSYQYKVALNDSWNSSYPSDNINLTVPDGGAHVTFSYVPSTHAVYDTINNPGANLPLDGSGIKTDLVTVTLGENPDVSHTLSIQTDGFKTGRVIPRNVLDFSQYYYSGEDLGNTYTKKATTFKVWAPTSTKVNVLLYNKAAGALTKTVPMKASGHGVWSVTVPQNLENWYYLYEVTGQGSTRTAVDPYATAIAPNGTRGMVVDLAKTNPTGWKSDKHMTPKNIEDEVIYEMHVRDFSIDSNSGMTNKGKYLALTEKGTKGPENVKTGVDSLKQLGITHVQLQPVFAFNSVDETDPTQYNWGYDPRNYNVPEGQYATDANGTTRIKEFKEMVLSLHRNHIGVNMDVVYNHTFATQISDFDKIVPQYYYRTDDAGNYTNGSGTGNEVAAERPMVQKFIIDSLKYWVNEYHIDGFRFDLMALLGKDTMAKAAQELHAIDPGIALYGEPWTGGTSALPTDQLLTKGVQKGMGVAVFNDNLRNGLDGNVFDASSQGFATGATGLTDVIKKGVEGSINDFTSSPGETINYVTSHDNYTLWDKIAQSNPNDSEADRIKMDELAQAVVVTSQGVPFMQGGEEMLRTKGGNSNSYNAGDAVNEFDWSRKAQYSDVFNYYSGLIHLRLAHPAFRMTTANQIKEHLQFIDSPDNTVAYELTNHANKDKWGNIVVIYNPNKTAETVNLPSGKWAINATNGKIGESTLSHAEGHVQVPGISMMILHQETNK

SEQ ID NO: 15 - protein, 928 aa, Bacillus deramificans
DGNTTTIIVHYFRPAGDYQPWSLWMWPKDGGGAEYDFNQPADSFGAVASADIPGNPSQVGIIVRTQDWTKDVSADRYIDLSKGNEVWLVEGNSQIFYNEKDAEDAAKPAVSNAYLDASNQVLVKLSQPLTLGEGASGFTVHDDTANKDIPVTSVKDASLGQDVTAVLAGTFQHIFGGSDWAPDNHSTLLKKVTNNLYQFSGDLPEGNYQYKVALNDSWNNPSYPSDNINLTVPAGGAHVTFSYIPSTHAVYDTINNPNADLQVESGVKTDLVTVTLGEDPDVSHTLSIQTDGYQAKQVIPRNVLNSSQYYYSGDDLGNTYTQKATTFKVWAPTSTQVNVLLYDSATGSVTKIVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDNVKTGIDSLKQLGITHVQLMPVFASNSVDETDPTQDNWGYDPRNYDVPEGQYATNANGNARIKEFKEMVLSLHREHIGVNMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPDDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDAVNEFDWSRKAQYPDVFNYYSGLIHLRLDHPAFRMTTANEINSHLQFLNSPENTVAYELTDHVNKDKWGNIIVVYNPNKTVATINLPSGKWAINATSGKVGESTLGQAEGSVQVPGISMMILHQEVSPDHGKK

SEQ ID NO: 16 - protein, 750 aa, Pseudomonas amyloderamosa
AINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISK

(19) United States
(12) Patent Application Publication
(10) Pub. No.: US 2009/0280553 A1
(43) Pub. Date: Nov. 12, 2009

(54) METHOD FOR DESIGNING MUTATED ENZYME, METHOD FOR PREPARING THE SAME, AND MUTATED ENZYME

(75) Inventors: Bunzo Mikami, Uji-shi (JP); Hiroyuki Iwamoto, Fukuyama-shi (JP); Shotaro Yamaguchi, Kakamigahara-shi (JP)

Correspondence Address: EDWARDS ANGELL PALMER & DODGE LLP, P.O. BOX 55874, BOSTON, MA, 02205, US

(73) Assignee: AMANO ENZYME INC., Nagoya-shi (JP)

(21) Appl. No.: 12/309,929

(22) PCT Filed: Jun. 28, 2007
(86) PCT No.: PCT/JP2007/062996

§ 371 Date: Jul. 7, 2009

(30) Foreign Application Priority Data
Aug. 4, 2006 (JP) 2006-213490

(51) Int. Cl. C12N 9/24; C12N 9/44; C12N 9/26; C07H 21/04; C12N 1/21

(52) U.S. Cl. 435/201; 435/200; 435/210; 536/23.2; 435/252.3; 435/252.33

(57) ABSTRACT

It is intended to provide a novel method for improving an enzyme hydrolyzing an a-1,6-glycosidic linkage. A mutated enzyme is designed by specifying one or more amino acids selected from the group shown below in an amino acid sequence of an enzyme (an enzyme to be mutated) that hydrolyzes an a-1,6-glycosidic linkage, that is, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid represented by in SEQ ID NO: 2 (step (1)) and constructing an amino acid sequence in which the amino acid(s) specified in the step (1) is/are substituted with another amino acid or deleted based on the amino acid sequence of the enzyme to be mutated (step (2)).

### TECHNICAL FIELD

The present invention relates to a method for designing a mutated enzyme hydrolyzing an α-1,6-glycosidic linkage, a method for preparing the same, and a mutated enzyme.

### BACKGROUND ART

Pullulanase (EC 3.2.1.41) is an enzyme hydrolyzing an α-1,6 linkage of, for example, amylopectin in starch. Pullulanase is an enzyme having a high industrial applicability in the fields of sugar, for example, production of maltooligosaccharides such as glucose, maltose, maltotriose, maltotetraose, maltopentaose and maltohexaose (OLIGOSACCHARIDES, Gordon and Breach Science Publishers, p 3), improvement of rice cooking (patent document 1), and the like.

Pullulanase derived from microorganism includes Bacillus sp. APC-9603 (patent document 2), and ones derived from Klebsiella pneumonia (AMANO ENZYME INC.), Bacillus deramificans, Bacillus acidpullulyticus, Bacillus stearothermophilus, Bacillus sectorramus, Bacillus circulans, Bacillus cereus, and Bacillus sectorramus.

Similar to the other enzymes, when pullulanase is used, concentration of substrate and enzyme, reaction temperatures, reaction time, and the like are adjusted depending upon the applications of use. However, with adjustment of such enzyme reaction conditions alone, it may not be possible to produce intended products or to obtain an expected yield. Thus, it has been necessary to modify the properties themselves of pullulanase.

In order to modify the properties of pullulanase, it is necessary that mutants of pullulanase should be produced, and the activity, substrate specificity, and the like, should be evaluated so as to search for an excellent mutant. However, such processes have required much labor. Patent document 3 discloses one example of a mutant of pullulanase.

[Patent document 1] JP H7-289186 A

[Patent document 2] JP H5-292962 A

[Patent document 3] JP 2002-505108 A

[Non-patent document 1] J Mol Biol. 2006 Jun. 9; 359 (3): 690-707

### DISCLOSURE OF THE INVENTION

### [Problems to be Solved by the Invention]

One of objects of the present invention is to provide a novel method for improving an enzyme hydrolyzing an α-1,6-glycosidic linkage. Another object of the present invention is to provide a mutated enzyme whose action properties have been improved. With the change in action property, it is possible to reduce the amount of enzyme to be used, to shorten a reaction time, to increase applications of use, and the like.

### [Means to Solve the Problems]

In order to solve the above-mentioned problems, the present inventors have keenly investigated further. As a result, the present inventors have obtained an important finding regarding the recognition of a substrate in pullulanase derived from Bacillus subtilis strain 168 by making good use of an X-ray analysis technology for a crystalline structure. That is to say, regarding the pullulanase, the present inventors have succeeded in crystallizing it into a state containing a substrate analog (α-cyclodextrin), and in obtaining information about the three-dimensional structure thereof. Thereby, they have clarified a site to which a substrate analog is bound. Thus, amino acid that is thought to be involved in recognition of a substrate has been specified. Furthermore, as a result of comparison between the three-dimensional structure of the pullulanase and the three-dimensional structure of the same kinds of enzymes derived from the other microorganisms, high similarity is recognized as a whole. In particular, it has been determined that the similarity is extremely high in the site relating to the recognition of a substrate. Since such a high similarity is recognized, it is predicted that an amino acid corresponding to the amino acid specified in the above-mentioned pullulanase plays an important role in the recognition of a substrate in each enzyme.

By the way, as to pullulanase of Klebsiella pneumoniae that is one of the enzymes used in the investigation at this time, an active site is searched for by using G4 (maltotetraose) (see, non-patent document 1). The binding site of the substrate indicated therein is located in the vicinity of the binding site of a substrate analog predicted by the above-mentioned method (a method by comparing with pullulanase derived from Bacillus subtilis strain 168). This fact supports the involvement of the binding site of the substrate analog successfully found by the present inventors in the recognition of actual substrate.

The present invention is mainly based on the above-mentioned results and provides a designing method of an enzyme mentioned below.

[1] A method for designing a mutated enzyme, the method including the following steps:

(1) specifying one or two or more amino acids selected from the group shown below in an amino acid sequence of an enzyme (enzyme to be mutated) that hydrolyzes an α-1,6-glycosidic linkage, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2; and

(2) constructing an amino acid sequence in which the amino acid specified in the step (1) is substituted with another amino acid or deleted based on the amino acid sequence of the enzyme to be mutated.

[2] The method for designing a mutated enzyme according to [1], wherein in the step (1), one or two or more amino acids selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position, and an amino acid corresponding to an amino acid at the 582 position of the amino acid sequence set forth in SEQ ID NO: 2 is specified.
[3] The method for designing a mutated enzyme according to [1], wherein in the step (1), an amino acid corresponding to an amino acid at the 476 position of an amino acid sequence set forth in SEQ ID NO: 2 is specified.
[4] The method for designing a mutated enzyme according to any one of [1] to [3], wherein the specifying of an amino acid in step (1) is carried out by comparing between an amino acid sequence of the enzyme to be mutated and the amino acid sequence set forth in SEQ ID NO: 2 and/or between a three-dimensional structure of the enzyme to be mutated and a three-dimensional structure of the amino acid sequence set forth in SEQ ID NO: 2.
[5] The method for designing a mutated enzyme according to any one of [1] to [4], wherein the enzyme to be mutated is a wild type enzyme.
[6] The method for designing a mutated enzyme according to any one of [I] to [4], wherein the enzyme to be mutated is pullulanase or isoamylase derived from a microorganism.
[7] The method for designing a mutated enzyme according to [6], wherein the microorganism is a microorganism of genus bacillus, a microorganism of genus Klebsiella, or a microorganism of genus pseudomonas.
[8] The method for designing a mutated enzyme according to any one of [1] to [4], wherein the amino acid sequence of the enzyme to be mutated is an amino acid sequence having a 70% or more identity to the amino acid sequence set forth in SEQ ID NO: 2.
[9] The method for designing a mutated enzyme according to any one of [1] to [4], wherein the amino acid sequence of the enzyme to be mutated is an amino acid sequence set forth in any of SEQ ID NOs:2, 13 to 16.
[10] A method for preparing a mutated enzyme, the method including the following steps:

(1) preparing a nucleic acid encoding an amino acid sequence constructed by the method described in any of [1] to [9];

(2) expressing the nucleic acid; and

(3) collecting expression products.

[11] A mutated enzyme including an amino acid sequence in which one or two or more amino acids selected from the group shown below in an amino acid sequence of an enzyme (enzyme to be mutated) that hydrolyzes an α-1,6-glycosidic linkage, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2 is/are substituted with another amino acid or deleted.
[12] The mutated enzyme according to [11], wherein the substituted or deleted amino acid is one or two or more amino acids selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position, and an amino acid corresponding to an amino acid at the 582 position of the amino acid sequence set forth in SEQ ID NO: 2.
[13] The mutated enzyme according to [11], wherein the substituted or deleted amino acid is an amino acid corresponding to an amino acid at the 476 position of the amino acid sequence set forth in SEQ ID NO: 2.
[14] The mutated enzyme according to any one of [11] to [13], wherein the enzyme to be mutated is a wild type enzyme.
[15] The mutated enzyme according to any one of [11] to [13], wherein the enzyme to be mutated is pullulanase or isoamylase derived from a microorganism.
[16] The mutated enzyme according to [15], wherein the microorganism is a microorganism of genus bacillus, a microorganism of genus Klebsiella, or a microorganism of genus pseudomonas.
[17] The mutated enzyme according to any of [11] to [13], wherein the amino acid sequence of the enzyme to be mutated is an amino acid sequence having a 70% or more homology with respect to the amino acid sequence set forth in SEQ ID NO: 2.
[18] The mutated enzyme according to any of [11] to [13], wherein the amino acid sequence of the enzyme to be mutated is an amino acid sequence set forth in any of SEQ ID NOs: 2, 13 to 16.
[19] The mutated enzyme according to any of [11] to [13], wherein an action property with respect to pullulan or an action property with respect to amylopectin is improved as compared with the enzyme to be mutated.
[20] A gene encoding the mutated enzyme according to any of [11] to [19].
[21] A recombinant DNA including the gene according to [20].
[22] A microorganism carrying the recombinant DNA according to [21].

### BRIEF DESCRIPTION OF THE DRAWINGS

FIG. 1 is a view showing a three-dimensional structure of Bacillus subtilis pullulanase having α-cyclodextrin as a ligand, which is shown by the use of a ribbon model. CD: α-cyclodextrin.

FIG. 2 is a view shown by superimposing Bacillus subtilis pullulanase (BSP) having α-cyclodextrin as a ligand and a carbon of pullulanase (KPP) Klebsiella pneumonia onto each other. CD: α-cyclodextrin.

FIG. 3 is an enlarged view showing a substrate binding region of FIG. 2. CD: α-cyclodextrin, G4: maltotetraose. An amino acid of Bacillus subtilis pullulanase (upper stage) and an amino acid of pullulanase of Klebsiella pneumonia (lower stage) corresponding to the amino acid of the upper stage are shown.

FIG. 4 is a view shown by superimposing Bacillus subtilis pullulanase (BSP) having α-cyclodextrin as a ligand and α carbon of isoamylase of Pseudomonas amyloderamosa (PIA) onto each other. CD: α-cyclodextrin.

FIG. 5 is an enlarged view showing a substrate binding region of FIG. 4. CD: α-cyclodextrin, G4: maltotetraose. An amino acid of Bacillus subtilis pullulanase (upper stage) and an amino acid of isoamylase of Pseudomonas amyloderamosa (lower stage) corresponding to the amino acid of the upper stage are shown.

FIG. 6 shows a multiple alignment of amino acid sequences of five kinds of pullulanase and isoamylase. BSP: pullulanase of Bacillus subtilis, BCP: pullulanase of Bacillus sp. APC-9603, BDP: pullulanase of Bacillus deramificans, KPP: pullulanase of Klebsiella pneumoniae, PIA: isoamylase of Pseudomonas amyloderamosa (Amemura, A., Chakraborty, R., Fujita, M., Noumi, T. and Futai, M., Cloning and nucleotide sequence of the isoamylase gene from Pseudomonas amyloderamosa SB-15, JOURNAL J. Biol. Chem. 263 (19), 9271-9275 (1988)). *: position of an amino acid that has been deduced to be involved in binding to α-cyclodextrin, +: amino acid that has been deduced to be involved in binding to G4 altotetraose) among amino acids that have been deduced to be involved in binding to α-cyclodextrin in the above-mentioned report, −: position of the amino acid that has been deduced to be involved in binding to α-cyclodextrin in the above-mentioned report (excluding the position of amino acid that has been deduced to be involved in binding to α-cyclodextrin). Pul/Iso (pullulanase/isoamylase) specific region, region I, region II, region III and region IV are shown by a shaded area. Furthermore, an amino acid of the active site is surrounded by square.

FIG. 7 is the continuation of FIG. 6.

FIG. 8 is an enlarged view showing an α-cyclodextrin biding site of FIG. 1.

### BEST MODE FOR CARRYING OUT THE INVENTION

### 1. Designing Method of Mutated Enzyme

A first aspect of the present invention provides a designing method of a mutated enzyme based on an enzyme hydrolyzing an α-1,6-glycosidic linkage. With the designing method of the present invention, it is possible to obtain an enzyme that is different from the enzyme before mutation in terms of action properties. In other words, the designing method of the present invention is used as a technique for changing the action properties of an enzyme. Specifically, for example, the designing method of the present invention can be used for the purpose of improving the activity and/or substrate specificity of pullulanase with respect to pullulan, or the activity and/or substrate specificity of pullulanase with respect to amylopectin. It can be expected that the improvement of the activity enables obtaining of a sufficient effect with less amount. That is to say, reduction of the amount to be used can be expected. On the other hand, the improvement of the substrate specificity facilitates the use thereof and reduces the amount to be used. Furthermore, if different substrate specificities are provided, a novel application of use can be achieved.

Pullulanase that is one of the enzymes hydrolyzing an α-1,6-glycosidic linkage can act on amylopectin in starch so as to form straight chain amylase. By the use of this characteristic, pullulanase has been widely used for processing starch, production of glucose, maltose, oligosaccharide, or the like, or brewing. Furthermore, pullulanase is an enzyme that is used for various industrial purposes of, for example, manufacturing a material of thermally stable microcapsule, a carrier of an immobilized enzyme, and the like. If the reactivity with respect to α-1,6 binding can be freely changed, for example, it is possible to increase the yield in products, to reduce the amount of enzyme to be used (an amount to be added). At the same time, this enzyme can be applied to new fields.

In the present specification, unless otherwise noted, the term “action property” is used as a term including properties (including activity and substrate specificity with respect to pullulan and activity and substrate specificity with respect to amylopectin) relating to the actions for hydrolyzing an α-1,6-glycosidic linkage. The evaluation of the “action property” can be carried out by using the Km value, Kcat value, and the like, obtained by the test system using pullulan, amylopectin, and starch as a substrate. Km value, Kcat value can be determined by the following method.

(1) Substrates (for example, pullulan or amylopectin) with various concentrations are dissolved in 50 mM acetate buffer (pH 5.6) and reacted at 25° C.

(2) The concentration of a reducing sugar contained in a regularly sampled reaction solution is determined by a Park-Johnson method, and the reaction rate is measured from the increasing rate of the reducing sugar.

(3) Km value and Kcat value are obtained by curve fitting into Michaelis-Menten equation by the non-linear minimum square method.

Note here that although depending upon the experiment conditions, by comparing the concentrations of the reducing sugar contained in the reaction solution at certain points, the action properties of two enzymes can be compared and evaluated.

The designing method of the mutated enzyme of the present invention includes roughly two steps, that is, a step of specifying an amino acid to be mutated (step (1)) and a step of constructing an amino acid sequence of the mutated amino acid (step (2)). Hereinafter, the respective steps are described in detail. Note here that in this specification, an enzyme as a base in designing a mutated enzyme (an enzyme to which mutation is carried out) is referred to as “enzyme to be mutated.”

### Step (1)

In step (1), in an amino acid sequence of an enzyme (enzyme to be mutated) hydrolyzing an α-1,6-glycosidic linkage, one or two or more of amino acid(s) to which mutation is carried out (hereinafter, which is also referred to as “amino acid to be mutated”) is specified. The amino acid to be mutated of the present invention is selected from the group shown below in an amino acid sequence of an enzyme that hydrolyzes an α-1,6-glycosidic linkage, that is, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2. Note here that these amino acids to be mutated are amino acids that have been suggested to be involved in the recognition of the substrate as a result of analysis of the three-dimensional structure at the time of binding of a substrate analog (α-cyclodextrin, hereinafter, referred to as “CD”) regarding pullulanase derived from Bacillus subtilis strain 168 (including the amino acid sequence set force in SEQ ID NO: 2), and from the comparison results between this three-dimensional structure and the three-dimensional structure of an enzyme derived from a microorganism. By mutating these amino acids, it is expected that the action property (in particular, substrate specificity) of the enzyme is changed.

Herein, the term “corresponding” to be used for amino acid residues in the specification means the equal contribution to exhibition of the function between proteins (enzymes) to be compared. In particular, it means that the contribution to the substrate specificity is equivalent. For example, when an amino acid sequence to be compared is arranged with respect to the reference amino acid sequence (that is to say, amino acid sequence set forth in SEQ ID NO: 2) so that suitable comparison can be carried out while considering the partial homology of the primary structure (that is to say, an amino acid sequence) (at this time, a gap may be introduced so as to optimize the alignment if necessary), an amino acid in a position corresponding to a certain amino acid in the reference to amino acid sequence can be defined as “corresponding amino acid.” Instead of comparison between primary structures, or in addition thereto, by comparison between the stereostructures (three-dimensional structures), “corresponding amino acid” can be specified. By using the three-dimensional structure information, it is possible to comparison results with high reliability. In this case, atomic coordinates of the three-dimensional structures of a plurality of enzymes can be compared with each other so as to carry out alignment. The three-dimensional structure information on the enzyme to be mutated can be obtained from, for example, Protein Data Bank (http://www.pdbj.org/index_j.html).

An example of the method of determining the three-dimensional structure of protein by an X-ray analysis of crystalline structure is described below.

(1) Protein is crystallized. The crystallization is indispensable for determination of the three-dimensional structure. Besides, the crystallization is industrially useful as a purification of protein with high purity and a preservation method of protein with high density. In this case, protein to which a substrate or an analog compound thereof is bound as a ligand may be crystallized.

(2) The prepared crystal is irradiated with X ray and analysis data are collected. Note here that protein crystal may be damaged by X ray irradiation and may be deteriorated in its diffraction ability so often. In such a case, a low-temperature measurement method for rapidly cooling a crystal to about −173° C. and collecting diffraction data in this state has been recently widespread. Note here that finally, in order to collect high resolution data used for determining a structure, synchrotron radiation light with high intensity is used.

(3) For carrying out analysis of a crystalline structure, phase information is necessary in addition to the diffraction data. When the crystalline structure of a related protein with respect to the intended protein is not known, it is impossible to determine the structure by a molecule substitution method. Problem as to the phase must be resolved by the heavy atom isomorphous replacement method. The heavy atom isomorphous replacement method is a method of introducing a metal atom having a larger atomic number such as mercury and platinum into a crystal and using the contribution of the metal atom to X-ray diffraction data of X-ray scattering power, thereby obtaining phase information. The determined phase can be improved by smoothing the electron density in the solvent region in the crystal. Since the water molecule in the solvent region is largely fluctuated, electrical density is hardly observed. Therefore, by approximating the electron density in this region to 0, it can approach to the real electron density. Consequently, the phase is improved. Furthermore, when a plurality of molecules are included in an asymmetrical unit, by averaging the electron densities of these molecules, the phase is further radically improved. A protein model is fitted to the view of the electron density calculated by using the thus improved phase. This process is carried out by using a program such as QUANTA (MSI, America) on a computer graphics. Thereafter, by using a program such as X-PLOR (MSI), refinement of the structure is carried out. Thus, the structure analysis is completed.

When the crystalline structure of a related protein with respect to the intended protein is known, the structure can be determined by a molecule substitution method by using an atomic coordinate of the known protein. The molecule substitution and structure refinement can be carried out by using a program such as CNS_SOLVE ver. 11.

The present inventors have tried to crystallize the recombinant pullulanase purified from Bacillus subtilis strain 168 and to crystallize the pullulanase in a state in which it contains CD as a substrate analog and have succeeded in obtaining three-dimensional structure of both types of pullulanase. Note here that atomic coordinates of the three-dimensional structure of the pullulanase containing CD are shown in the last part of this specification. Furthermore, the amino acid sequence of pullulanase and the base sequence of a gene encoding thereof are shown in SEQ ID NO: 2 and SEQ ID NO: 1 in the sequence listing, respectively.

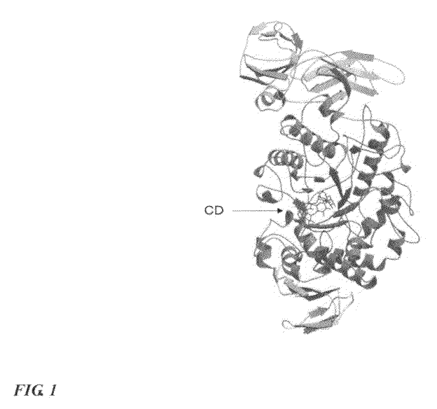

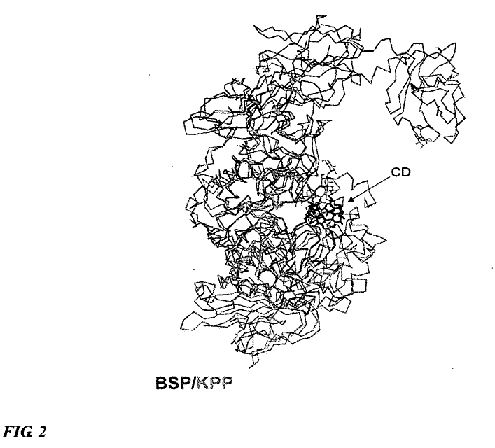

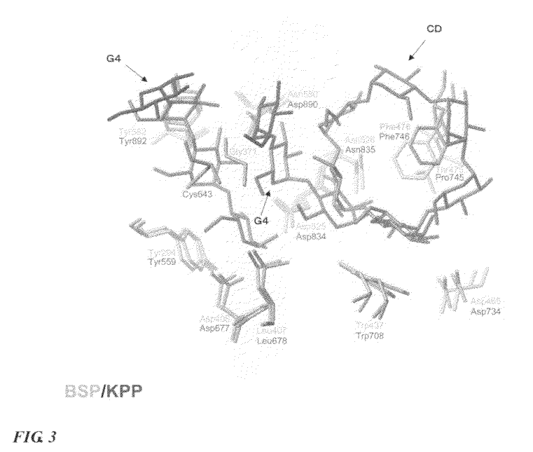

As shown in the below-mentioned Examples, it has been determined that the pullulanase molecule derived from Bacillus subtilis strain 168 has rhombic system P2(1)2(1)2(1) having 70.568×127.68×189.25 Å (see, FIGS. 1 to 3). FIG. 1 is a view showing a crystalline structure of pullulanase by a ribbon model. α-helix and β-sheet are shown in a helix shape and an arrow shape, respectively (FIG. 1), and a substrate analog (CD) is shown by an arrow CD (FIGS. 1 to 3). FIG. 2 is a view shown by superimposing Bacillus subtilis pullulanase (BSP) having α-cyclodextrin as a ligand and α carbon of pullulanase (KPP) Klebsiella pneumonia onto each other. FIG. 3 is an enlarged view of a substrate binding region of FIG. 2.

In one preferable embodiment of the present invention, the amino acid to be mutated is selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position and an amino acid corresponding to an amino acid at the 582 position of the amino acid sequence set forth in SEQ ID NO: 2. Note here that amino acids to be mutated are an amino acid corresponding to an amino acid that has been determined to be directly involved in binding between pullulanase derived from Bacillus subtilis strain 168 and a substrate analog (CD).

By the way, it has clarified that an amino acid at the 476 position of the amino acid sequence set forth in SEQ ID NO: 2 is arranged so that it enters a ring structure of CD that is a substrate analog in the three-dimensional structure analysis about pullulanase derived from Bacillus subtilis strain 168, and this amino acid is thought to play an important role in the recognition of a substrate. Therefore, in the further preferable embodiment of the present invention, an amino acid corresponding to the amino acid is to be an amino acid to be mutated.

The kinds, origins and the like of enzymes to be mutated in accordance with the present invention are not particularly limited as long as the enzymes hydrolyze an α-1,6-glycosidic linkage. Preferably, the enzyme to be mutated is pullulanase or isoamylase derived from microorganisms. An example of the microorganism herein can include a microorganism of genus bacillus, a microorganism of genus Klebsiella, or a microorganism of genus pseudomonas. As the pullulanase derived from microorganism, Bacillus sp. APC-9603 (Japanese Patent Unexamined Publication No. H5-292962), and ones derived from derived from Klebsiella pneumonia (AMANO ENZYME INC.), Bacillus deramificans, Bacillus acidpullulyticus, Bacillus stearothermophilus, Bacillus sectorramus, Bacillus circulans, Bacillus cereus, and Bacillus subtilis strain 168 are well known. For example, any of them can be employed as the enzyme to be mutated in the present invention. Furthermore, isoamylase of Pseudomonas amyloderamosa can be employed as the enzyme to be mutated in the present invention. A specific example of the enzyme to be mutated includes an enzyme (pullulanase of Bacillus subtilis strain 168) consisting of an amino acid sequence set forth in SEQ ID NO: 2, an enzyme (pullulanase of Klebsiella pneumoniae ATCC9621) consisting of an amino acid sequence set forth in SEQ ID NO: 13, an enzyme (pullulanase of Bacillus sp. APC-9603) consisting of an amino acid sequence set forth in SEQ ID NO: 14, an enzyme (pullulanase of Bacillus deramificans) consisting of an amino acid sequence set forth in SEQ ID NO: 15, and an enzyme (isoamylase of Pseudomonas amyloderamosa) consisting of an amino acid sequence set forth in SEQ ID NO: 16.

It is preferable that an enzyme consisting of an amino acid sequence having a high identity with the amino acid sequence set forth in SEQ ID NO: 2 is an enzyme to be mutated. It is preferable because effective improvement is expected to be achieved and the specification of the amino acid to be mutated is facilitated.

Specifically, it is preferable that the enzyme to be mutated is an enzyme consisting of an amino acid sequence having 70% or more identity with the amino acid sequence set forth in SEQ ID NO: 2. Herein, the higher identity is generally more preferable. For example, the enzyme to be mutated is an enzyme consisting of an amino acid sequence having the identity of preferably 80% or more, further preferably 90% or more, and further preferably 95% or more.

Herein, the identity (%) between two amino acid sequences can be determined by the following procedure. Firstly, two sequences are aligned for optimum comparison of the two sequences (for example, a gap may be introduced in the first sequence so as to obtain an optimum alignment with the second sequence). When a molecule (amino acid residue) at the specific position in the first sequence and a molecule in the corresponding position in the second sequence are the same, the molecules in the positions are defined as being identical. The identity between two sequences is an action property of the number of identical positions shared by the sequences (i.e., identity (%)=number of identical positions/total number of positions×100), preferably taking into account the number of gaps, and the length of each gap, which need to be introduced for optimal alignment of the two sequences.

The comparison and determination of identity between two sequences can be carried out by using a mathematical algorithm. A specific example of mathematical algorithm that can be used for comparing sequences include an algorithm described in Karlin and Altschul (1990) Proc. Natl. Acad. Sci. USA 87:2264-68 and modified by Karlin and Altschul (1993) Proc. Natl. Acad. Sci. USA 90:5873-77 but the algorithm is not limited to this. Such an algorithm is incorporated in NBLAST and XBLAST programs (version 2.0) of Altschul et al. (1990) J. Mol. Biol. 215: 403-10. BLAST polypeptide searches may be carried out by, for example, the NBLAST program, score=50, wordlength=3 to obtain amino acid sequence homologous to a certain amino acid sequence. To obtain gapped alignments for comparison purposes, Gapped BLAST as described in Altschul et al., (1997) Amino Acids Research 25(17): 3389-3402 can be utilized. When utilizing BLAST and Gapped BLAST programs, the default parameters of the respective programs (e.g., XBLAST and NBLAST) can be used. In detail, see http://www.ncbi.nlm.nih.gov. Another example of mathematical algorithm that can be used for comparing sequences includes an algorithm of Meyers and Miller (Comput. Appl. Biosci. 4: 11-17 (1988)) which has been incorporated into the ALIGN program that can be used for, for example, GENESTREAM network server (IGH Montpellier, France) or ISREC server. When the ALIGN program is used for comparison of the amino acid sequences, for example, a PAM120 weight residue table can be used with a gap length penalty of 12 and a gap penalty of 4.

The identity between two amino acid sequences can be determined using the GAP program in the GCG software package, using a Blossom 62 matrix or PAM250 matrix and a gap weight of 12, 10, 8, 6, or 4, and a gap length weight of 2, 3, or 4.

Furthermore, the homology between two nucleic acid sequences can be determined using the GAP program in the GCG software package (available at http://www.gcg.com) with a gap weight of 50 and a gap length weight of 3.

The enzyme to be mutated is typically a wild type enzyme (naturally occurring enzyme). However, an enzyme to which some mutation or modification has already been given is not excluded. Thus, the present invention can be used for the purpose of further improving the property of an enzyme.

### Step (2)

In the step (2), an amino acid sequence, in which an amino acid specified in the step (1) has been substituted with another amino acid or the amino acid has been deleted, is constructed based on an amino acid sequence of the enzyme to be mutated. The kinds of substituted amino acids are not particularly limited and therefore may include conservative substitution of amino acid or non-conservative substitution of amino acid. Herein, a “conservative amino acid substitution” is one in which the amino acid residue is substituted with an amino acid residue having a side chain with similar feature. The amino acid residues are divided into some families including basic side chains (e. g., lysine, arginine, histidine), acidic side chains (e.g., aspartic acid, glutamic acid), uncharged polar side chains (e. g., asparagine, glutamine, serine, threonine, tyrosine, cysteine), nonpolar side chains (e. g, glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). Preferably, the conservative amino acid substitution is a substitution between preferably an amino acid residue of the same family.

### 2. Preparation Method of Mutated Enzyme

A second aspect of the present invention relates to a preparation method of a mutated enzyme. The preparation method in accordance with the present invention includes the following steps:

(1) preparing nucleic acid encoding an amino acid sequence constructed by the designing method in accordance with the present invention;

(2) expressing the nucleic acid; and

(3) collecting expression products.

In the step (1), necessary mutation (that is, substitution or deletion of amino acids in a certain position in protein as an expression product) is applied to a gene encoding the enzyme to be mutated based on an amino acid sequence constructed by the designing method of the present invention, and thereby nucleic acid (gene) encoding a mutated enzyme is obtained. A large number of methods for the position specific substitution of base sequence have been known (see, for example, Molecular Cloning, Third Edition, Cold Spring Harbor Laboratory Press, New York). Among them, appropriate methods can be selected and used.

As the position specific mutation introduction method, a position specific amino acid saturated mutation method can be employed. The position specific amino acid saturated mutation method is a “Semi-rational, semi-random” technique in which a position relating to the intended function is deduced based on the three-dimensional structure of protein, and the amino acid saturated mutation is introduced (J. Mol. Biol. 331, 585-592 (2003)). For example, a position specific amino acid saturated mutation can be introduced by using a kit such as Quick change (Stratagene), Overlap extension PCR (Nucleic Acid Res. 16, 7351-7367 (1988)). As DNA polymerase used for PCR, Taq polymerase can be used. However, it is preferable that DNA polymerase with high purify, for example, KOD-PLUS-(TOYOBO), Pfu turbo (Stratagene) are used.

On the other hand, a gene encoding a mutated enzyme can be prepared by inserting random mutation into an enzyme gene, comparing the substrate specificities of expression product by mutants each other, and selecting a gene having preferable substrate specificity. When such a random mutation is introduced, firstly, for example, error-prone PCR is used and mutation is introduced into a targeted gene region randomly so as to construct a mutated enzyme gene library. Then, a clone is selected from the resultant library using the enzymatic activity or the substrate specificity as an index.

In the step (2), a gene prepared in the step (1) is expressed. Then, in the subsequent step (3), mutated enzymes as expression products are collected.

In general, from the step of expressing a gene to the step of collecting the expression products (mutated enzymes) are carried out by using an appropriate host-vector system. However, a cell-free synthesis system may be used. As to the detail of the preparation method of the mutated enzyme by using a host-vector system, the below-mentioned description may be employed (see, the column of 4. Nucleic acid encoding mutated enzyme).

Herein, the “cell-free synthesis system (cell-free transcription system, cell-free transcription/translation system)” denotes that living cells are not used but a ribosome derived from living cells (or cells obtained by genetically engineering technique) or by using a transcription/translation factor and the like, mRNA or protein encoded by nucleic acid (DNA or mRNA) as a template are synthesized from them in vitro. In general, in the cell-free synthesis system, a cell extract obtained by purifying a cell homogenized solution if necessary is used. In general, a cell extract includes ribosome necessary to synthesis of protein, various factors such an initiation factor, various enzymes such as tRNA. When synthesis of protein is carried out, various amino acids, energy sources such as ATP and GTP, creatine phosphate, and the like, are added to the cell extract solution. Needless to say, at the time of synthesis of protein, additionally prepared ribosome or various factors, and/or various enzymes may be replenished if necessary.

Development of a transcription/translation system in which each molecule (factor) necessary to synthesis of protein is reconstructed has been reported (Shimizu, Y. et al.: Nature Biotech., 19, 751-755, 2001). In this system, a gene of 31 kinds of factors consisting of three kinds of initiation factors constituting a protein synthesis system of bacteria, three kinds of elongation factors, four kinds of factors involved in termination, 20 kinds of aminoacyl tRNA synthases for binding each amino acid to tRNA, and methionyl tRNA formyl transferase is amplified from Escherichia coli genome. They are used so as to reconstruct a protein synthesis system in vitro. In the present invention, such a re-constructed synthesis system may be used.

The term “cell-free transcription/translation system” can be used interchangeably with the term cell-free protein synthesis system, in vitro translation system or in vitro transcription/translation system. In the in vitro translation system, protein is synthesized by using RNA as a template. As the template RNA, total RNA, mRNA, in vitro transcription product, and the like, are used. On the other hand, in the in vitro transcription/translation system, DNA is used as a template. The template DNA should include a ribosome-binding region and preferably includes an appropriate terminator sequence. Note here that the in vitro transcription/translation system sets a condition in which factors necessary to reaction are added so that the transcription reaction and translation reaction proceed consecutively.

### 3. Mutated Enzyme

According to the above-mentioned preparation method, it is possible to obtain a mutated enzyme in which the action property with respect to α-1,6-glycosidic linkage has been changed. Then, a further aspect of the present invention provides a mutated enzyme. In the mutated enzyme of the present invention, an action property with respect to pullulan or action property with respect to amylopectin are improved over the enzyme to be mutated.

The mutated enzyme of the present invention is an amino acid sequence wherein in the amino acid sequence of enzyme (enzyme to be mutated) hydrolyzing an α-1,6-glycosidic linkage, one or two or more amino acids selected from the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2 is/are substituted with another amino acid or deleted.

Preferably, the substituted or deleted amino acid is one or two or more amino acid selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position, and an amino acid corresponding to an amino acid at the 582 position of the amino acid sequence set forth in SEQ ID NO: 2.

Further preferably, the substituted or deleted amino acid is an amino acid corresponding to an amino acid at the 476 position of the amino acid sequence set forth in SEQ ID NO: 2.

The kinds, origins and the like of the enzymes to be mutated are the same as those in the above-mentioned first aspect, and therefore the same description is omitted herein. A specific example of the enzyme to be mutated includes an enzyme (pullulanase of Bacillus subtilis strain 168) consisting of an amino acid sequence set forth in SEQ ID NO:2, an enzyme (pullulanase of Klebsiella pneumoniae ATCC9621) consisting of an amino acid sequence set forth in SEQ ID NO: 13, an enzyme (pullulanase of Bacillus sp. APC-9603) consisting of an amino acid sequence set forth in SEQ ID NO: 14, an enzyme (pullulanase of Bacillus deramificans) consisting of an amino acid sequence set forth in SEQ ID NO: 15, and an enzyme (isoamylase of Pseudomonas amyloderamosa) consisting of an amino acid sequence set forth in SEQ ID NO: 16.

The mutated enzyme of the present invention is characterized by having an amino acid sequence in which a certain position of the amino acid sequence of the enzyme before mutation (enzyme to be mutated) has been mutated. In a position other than the position related to the mutation, a part of the amino acid may be mutated or modified. Thus, the present invention also provides protein having the same function as compared with the mutated enzyme having the amino acid sequence to which the above-mentioned mutation has been given but having a difference in a part of the amino acid sequence (hereinafter, also referred to as “homologous protein”). The term “having a difference in a part of the amino acid sequence” typically means that the amino acid sequence is mutated (changed) by the deletion and substitution of one to several amino acids constituting the amino acid sequence, or addition of one to several amino acids, or the combination thereof. The difference in the amino acid sequence herein is acceptable as long as the properties related to hydrolysis of an α-1,6-glycosidic linkage are not radically reduced (preferably, in the limit substantially held). As long as this condition is satisfied, the position of difference in the amino acid sequence is not particularly limited and the difference may occur in a plurality of positions. The plurality herein signifies a numerical value corresponding to less than about 30%, preferably less than about 20%, further preferably less than about 10%, yet further preferably less than about 5%, and most preferably less than about 1% with respect to the entire amino acid. That is to say, homologous protein has, for example, about 70% or more, preferably about 80% or more, further preferably about 90% or more, yet further preferably about 95% or more and most preferably about 99% or more of similarly to the amino acid sequence of the mutated enzyme.

The mutated enzyme can be used for arbitrary applications that need hydrolyzing an α-1,6-glycosidic linkage. For example, the mutated enzyme can be used for production of maltooligosaccharide such as glucose, maltose, maltotriose, maltotetraose, maltopentaose, and maltohexaose, and for the improvement of rice cooking, and the like. The amount of the mutated enzyme to be used can be appropriately set so that the intended effect can be exhibited. For example, the enzyme reaction can be carried out under conditions so that the enzyme concentration in the reaction solution becomes about 10 nM to about 100 μM.

### 4. Nucleic Acid Encoding Mutated Enzyme

The present invention further provides a nucleic acid related to a mutated enzyme of the present invention. That is to say, the present invention provides a gene encoding a mutated enzyme, nucleic acid that can be used as a probe for identifying a nucleic acid encoding a mutated enzyme, and nucleic acid that can be used as a primer for amplifying or mutating a nucleic acid encoding the mutated enzyme.

A gene encoding a mutated enzyme is typically used for preparing a mutated enzyme. A genetically engineering preparation method using a gene encoding a mutated enzyme makes it possible to obtain a mutated enzyme in a more homogeneous state. Furthermore, the method is a suitable for preparing a large amount of mutated enzymes. Note here that the application of the gene encoding a mutated enzyme is not limited to preparation of a mutated enzyme. For example, the nucleic acid can be used as an research tool for the purpose of elucidating the mechanism of action of a mutated enzyme, or as a tool for designing or preparing a further mutant of an enzyme.

In the present invention, the “gene encoding a mutated enzyme” refers to nucleic acid capable of obtaining the mutated enzyme when it is expressed, and includes not only a nucleic acid having a base sequence corresponding to an amino acid sequence of the mutated enzyme but also a nucleic acid obtained by adding a sequence that does not encode the amino acid sequence to the nucleic acid. Furthermore, degeneracy of codon is also considered.

The nucleic acid of the present invention can be prepared in an isolated state by standard genetic engineering technique, molecular biological technique, biochemical technique, and the like, with reference to sequence information disclosed in this specification or attached sequence list.

Another embodiment of the present invention provides nucleic acid that has a base sequence having the same function as the base sequences of a gene encoding a mutated enzyme of the present invention but having a difference in a part of the base sequence (hereinafter, which is also referred to as “homologous nucleic acid.” Furthermore, a base sequence specifying the homologous nucleic acid is also referred to as a “homologous base sequence”). An example of the homologous nucleic acid can include DNA encoding a protein including a base sequence in which one or a plurality of bases are substituted, deleted, inserted, added or inverted relative to the base sequences of a gene encoding a mutated enzyme of the present invention and having an activity capable of hydrolyzing an α-1,6-glycosidic linkage. Such substitution, deletion, or the like, may be occurred in a plurality of sites. The “plurality” herein differs depending upon the position or kinds of amino acid residues in a three-dimensional structure of a protein encoded by the nucleic acid codes, but the “plurality” of bases includes, for example, 2 to 40 bases, preferably 2 to 20 bases and more preferably 2 to 10 bases.

The above-mentioned homologous nucleic acid can be obtained by introduction of mutation by, for example, a treatment with a restriction enzyme; treatment with exonuclease, DNA ligase, etc; introduction of mutation by a site-directed mutagenesis (Molecular Cloning, Third Edition, Chapter 13, Cold Spring Harbor Laboratory Press, New York); random mutagenesis (Molecular Cloning, Third Edition, Chapter 13, Cold Spring Harbor Laboratory Press, New York), and the like. Furthermore, homologous nucleic acid can be obtained by other methods such as irradiation with ultraviolet ray.

A further embodiment of the present invention relates to nucleic acid having a base sequence complementary to the base sequence of the gene encoding a mutated enzyme of the present invention. A further embodiment of the present invention provides nucleic acid having a base sequence at least 60%, 70%, 80%, 90%, 95%, 99% or 99.9% identical to the base sequences of the gene encoding a mutated enzyme of the present invention.

A further embodiment of the present invention relates to nucleic acid having a base sequence that hybridizes, under stringent conditions, to the base sequence complementary to the base sequences of the gene encoding a mutated enzyme of the present invention or the homologous base sequence thereof. The “stringent conditions” herein denote a condition in which a so-called specific hybrid is formed and a non-specific hybrid is not formed. Such stringent conditions are well known to the person skilled in the art and can be set with reference to Molecular Cloning (Third Edition, Cold Spring Harbor Laboratory Press, New York) or Current protocols in molecular biology (edited by Frederick M. Ausubel et al., 1987). An example of the stringent conditions includes a condition in which a DNA is incubated in a hybridization solution (50% formamide, 10×SSC (0.15 M NaCl, 15 mM sodium citrate, pH 7.0), 5×Denhardt solution, 1% SDS, 10% dextran sulfate, 10 μg/ml denatured salmon sperm DNA, 50 mM phosphate buffer (pH 7.5)) at about 42° C. to about 50° C., followed by washing with 0.1×SSC and 0.1% SDS at about 65° C. to about 70° C. A more preferable example of the stringent conditions can include a condition using a hybridization solution (50% formamide, 5×SSC (0.15 M NaCl, 15 mM sodium citrate, pH 7.0), 1×Denhardt solution, 1% SDS, 10% dextran sulfate, 10 μg/ml denatured salmon sperm DNA, 50 mM phosphate buffer (pH 7.5)).

A further embodiment of the present invention provides a base sequence of the gene encoding a mutated enzyme of the present invention or a nucleic acid (a nucleic acid fragment) having a part of a base sequence complementary to the base sequence. Such a nucleic acid fragment can be used for detecting, identifying and/or amplifying the nucleic acid having a base sequence of a gene encoding a mutated enzyme of the present invention. The nucleic acid fragment is designed to include at least a part for hybridizing a continuous nucleotide part (for example, about 10 to about 100 base length, preferably about 20 to about 100 base length, and further preferably about 30 to about 100 base length) in the base sequence of the gene encoding a mutated enzyme of the present invention.

When the nucleic acid fragment is used as a probe, it can be labeled. Labeling can be carried out by using a fluorescence material, enzyme, and radioactive isotope.

A yet further aspect of the present invention relates to recombinant DNA including a gene of the present invention (a gene encoding a mutated enzyme). The recombinant DNA is provided in a form of, for example, a vector. The term “vector” in this specification refers to a nucleic acid molecule that can transport a nucleic acid inserted therein to the inside of a target such as a cell.

In accordance with the purpose of use (cloning, protein expression), and by considering the kinds of host cells, an appropriate vector is selected. Specific examples of a vector include a vector using Escherichia coli as a host (M13 phage or the modified body thereof, λ phage or the modified body thereof, pBR322 or the modified body thereof (pB325, pAT153, pUC8, etc.) and the like), a vector using yeast as a host (pYepSec1, pMFa, pYES2, etc.), a vector using insect cells as a host (pAc, pVL, etc.), a vector using mammalian cells as a host (pCDM8, pMT2PC, etc.).

The vector of the present invention is preferably an expression vector. The term “expression vector” is a vector capable of introducing the nucleic acid inserted therein into the target cells (host cells) and expressing in the cells. The expression vector usually includes a promoter sequence necessary for expression of the inserted nucleic acid and an enhancer sequence for promoting the expression, and the like. An expression vector including a selection marker can be used. When such an expression vector is used, by using the selection marker, the presence or absence of the introduction of an expression vector (and the degree thereof) can be confirmed.

Insertion of the nucleic acid of the present invention into a vector, insertion of the selection marker gene (if necessary), and insertion of a promoter (if necessary), and the like, can be carried out by a standard recombination DNA technology (see, for example, Molecular Cloning, Third Edition, 1.84, Cold Spring Harbor Laboratory Press, New York, a well-known method using restriction enzyme and DNA ligase).

As the host cell, from the viewpoint of ease in handling, it is preferable that a bacterial cell such as Escherichia coli is used. However, the host cell is not limited to the bacterial cell as long as it can reproduce the recombinant DNA and express a gene of the mutated enzyme. As a preferable example of the host includes T7 system promoter, Escherichia coli BL21 (DE3) pLysS can be used. In other case, Escherichia coli JM109 can be used.

A further aspect of the present invention relates to a microorganism (that is, transformant) carrying a recombinant DNA of the present invention. The microorganism of the present invention can be obtained by the transfection or transformation using the above-mentioned vector of the present invention. For example, a calcium chloride method (J. Mol. Biol., Vol. 53, page 159 (1970)), a Hanahan method (J. Mol. Biol., Vol. 166, page 557 (1983)), a SEM method (Gene, Vol. 96, page 23 (1990)), a method by Chung et al. (Proceeding of the national Academy of Sciences of the USA, Vol. 86, page 2172 (1989)), a calcium phosphate coprecipitation method, electroporation (Potter, H. et al., Proc. Natl. Acad. Sci. U.S.A. 81, 7161-7165 (1984)), lipofection (Felgner, P. L. et al., Proc. Natl. Acad. Sci. U.S.A. 84, 7413-7417 (1984)), and the like.

A microorganism of the present invention can be used for producing a mutated enzyme of the present invention. That is to say, a further aspect of the present invention provides a method for producing a mutated enzyme of the present invention by using the above-mentioned microorganism. The production method of the present invention includes at least a step of culturing the above-mentioned microorganism under the conditions in which the mutated enzyme of the present invention is produced. In general, in addition to this step, a step of collecting (separating and purifying) the produced protein is carried out.

The microorganism (transformant) in accordance with the present invention is cultured in a usual manner. As a carbon source to be used for a medium, a carbon compound that can be assimilated may be used. An example may include glucose, sucrose, lactose, maltose, syrup, pyruvic acid, and the like. Furthermore, as a nitrogen source, any available nitrogen compounds may be used. An example may include peptone, meat extract, yeast extract, casein hydrolysate, soybean cake alkali extract, and the like. Besides, salts of phosphate, carbonate, sulfate, magnesium, calcium, potassium, iron, manganese, zinc, and the like, certain amino acids, certain vitamins, can be used if necessary.

On the other hand, the culture temperature can be set 30 to 40° C. (preferably about 37° C.). Culture time can be set considering the growing property of transformant to be cultured or production property of mutated enzyme, and the like. The pH of the medium is adjusted in a range in which the transformant is grown and an enzyme is produced. Preferably, pH of the medium is adjusted to about 6.0 to 9.0 (preferably, around pH 7.0).

A culture solution containing a bacterial body producing a mutated enzyme can be used as an enzyme solution as it is or after concentration or removing of impurities are carried out. Generally, however, a mutated enzyme is once collected from a culture solution or a bacterial body. When the produced mutated enzyme is secreting type protein, the mutated enzyme can be collected from the culture solution, and in other case, the mutated enzyme can be collected from the bacterial body. When the mutated enzyme is collected from the culture solution, purified products of the mutated enzyme can be obtained by, for example, subjecting the culture supernatant to filtering or centrifugation so as to remove insoluble matters, followed by carrying out separation and purification by the combination of vacuum concentration, membrane concentration, salting out using ammonium sulfate and sodium sulfate, fractional precipitation by methanol, ethanol or acetone, dialysis, heat treatment, isoelectric point treatment, various chromatography such as gel filtration chromatography, adsorption chromatography, ion exchange chromatography and affinity chromatography (for example, gel filtration by Sephadex gel (Pharmacia Biotech) and the like, DEAE sepharose CL-6B (Pharmacia Biotech), octyl sepharose CL-6B (Pharmacia Biotech), CM sepharose CL-6B (Pharmacia Biotech)), and the like. On the other hand, when the mutated enzyme is collected from a bacterial body, purified products of the mutated enzyme can be obtained by subjecting a culture solution to filtration and centrifugation to collect a bacterial body, followed by destructing the bacterial body by mechanical method such as pressure treatment and ultrasonication, or by an enzymatic method using lysozyme, and then carrying out separation and purification as mentioned above.

Purified enzyme obtained as mentioned above can be provided in a state of powder by, for example, freeze drying, vacuum drying or spray drying. At this time, a purified enzyme may be solved in phosphate buffer, triethanolamine buffer, Tris hydrochloric acid buffer, GOOD buffer in advance. Preferably, phosphate buffer and triethanolamine buffer can be used. An example of the GOOD buffer may include PIPES, MES or MOPS.

Hereinafter, the present invention is described further specifically. However, the present invention is not limited to these Examples.

### EXAMPLE

1. Preparation of Bacillus subtilis Recombinant Pullulanase
(1) Cloning of Bacillus subtilis Pullulanase Gene

A gene AmyX (GenBank Accession No. NC 000964) encoding pullulanase, which is found in the genome sequence of Bacillus subtilus, was amplified by PCR as follows. A chromosome DNA of Bacillus subtilis strain 168 isolated by the method by Sambrook et al. (Molecular Cloning: a laboratory manual, 2nd Edition, Cold Spring harbor Laboratory Press, 1989) was used as a template of PCR, and oligonucleotides of SEQ ID NO: 3 and SEQ ID NO: 4 were synthesized to form a primer. In the PCR reaction, 30 cycles of reactions of 94° C./2 minutes, 94° C./15 seconds-60° C./30 seconds and amplification of 68° C./4 minutes were carried out by using KOD plus system (TOYOBO). The obtained PCR fragment was treated with restriction enzymes NcoI and XhoI, and then linked to plasmid pET21d (Novagen), which had been cleaved with the both restriction enzymes. Thus, an expression plasmid pEBSP was obtained. It was confirmed by determining the base sequence that the obtained PCR fragment encoded pullulanase correctly.

SEQ ID NO: 3

5′-GGCCATGGTCAGCATCCGCCGCAGCTTCGA-3′
(underlined part represents a restriction enzyme
NcoI recognition site)

SEQ ID NO: 4

5′-GGCTCGAGTCAAGCAAAACTCTTAAGATCT-3′
(underlined part represents a restriction enzyme
XhoI recognition site)

(2) Expression of Bacillus subtilis Recombinant Pullulanase

The above-mentioned expression plasmid was introduced into Escherichia coli HMS174 (DE3) (Novagen) by transformation. The obtained transformant was inoculated on LB medium (500 ml) containing 100 μ/ml ampicillin and the medium was shaken at 37° C. At the time when the turbidity at 600 nm reached 0.6 to 0.8, isopropylthiogalactoside (IPTG) was added so that the final concentration became 0.5 mM and further cultured at 18° C. for 60 hours. The bacterial bodies were collected from the culture solution by centrifugation and suspended in a buffer solution (20 mM tris-hydrochloric acid (pH 8.1)/5 mM EDTA/20 mM β-mercaptoethanol/0.2 mM PMSF).

(3) Purification of Bacillus subtilis Recombinant Pullulanase

The suspension obtained in (2) was subjected to ultrasonication (4° C., 30 minutes, 200 μA) and then subjected to centrifugation (14000 rpm, 4° C., 30 minutes) to obtain a crude solution. To this solution, 30% saturated ammonium sulfate was added. Then, impurities were removed by centrifugation. To this centrifuged supernatant, 60% saturated ammonium sulfate was added. Then, precipitates were collected by centrifugation. The precipitates were dissolved in the buffer solution of (2) containing 20% saturated ammonium sulfate, and subjected to Butyl-Toyopearl 650M column (TOSOH CORPORATION) that had been equilibrated by the buffer solution containing 20% saturated ammonium sulfate, and eluted at the concentration gradient of 20% to 0% of ammonium sulfate. The obtained pullulanase active fraction was dialyzed to buffer solution of (2), and subjected to HiLoad Q Sepharose Column (Amersham) that had been equilibrated by the same buffer solution, and eluted at the concentration gradient of 0 to 0.5 M of NaCl to obtain active fraction. This fraction was dialyzed to the buffer solution of (2), and subjected to Mono Q Column (Amersham) that had been equilibrated by the same buffer solution, and eluted at the concentration gradient of 0 to 0.5 M of NaCl to obtain purified Bacillus subtilis recombinant pullulanase. The analysis result of N-terminal amino acid sequence of this purified enzyme was MVSIRRSFEA and completely identity to gene sequence.

2. X-ray Analysis of Bacillus subtilis Pullulanase

### (1) Crystallization

Crystallization of Bacillus subtilis recombinant pullulanase was carried out by the following procedure. Firstly, screening was carried out by a sitting drop vapor diffusion method by using a 96-well plate (product by Emerald Biostructure and Hampton Research). After 24 hours at 20° C., small and thin crystal was observed in Wizard II No. 8 well. Next, by changing the molecular weight and concentration of polyethylene glycol (PEG) or the concentration of buffer solution or salts, conditions were optimized. Finally, a crystal was obtained by a hanging drop vapor diffusion method by using a 24-well plate. The hanging drop (6 μl) consisting of 3μl of enzyme solution (10 mg/ml) and 3 μl of reservoir solution (10% PEG4000, 0.1 M acetate buffer, pH 5.2, 0.2 M Mg CH₃COO)₂) was used and incubated for one to two days. Thus, diamond shaped crystal was obtained. Prior to the X-ray analysis, treatment with a reservoir solution containing 30% glycerol was carried out and then cooled instantly by using liquid nitrogen (−173° C.).

### (2) X-ray Analysis

X-ray diffraction data were collected at a temperature of liquid nitrogen by using Synchrotron radiation BL-38BI (SPring-8, Hyogo, Japan) and processed by using HKL2000 program. X-ray diffraction data of 2.1 Å resolution were collected so as to determine the crystallographic parameter. Space group was P2₁2₁2₁ and lattice constant was a=70.57 Å, b=127.68 Å, and c=189.25 Å.

### (3) Determination of Three-dimensional Structure

A three-dimensional structure was determined at the resolution of 2.1 Å by a molecule substitution method using an atomic coordinate (PDB accession code 2FGZ) of pullulanase of Klebsiella pneumonia. The substitution of molecules and the refinement of structure were carried out by using program CNS_SOLVE ver. 11. Data related to statistics of data collection and refinement are shown in Table 1.

TABLE 1

| Crystal | Bacillus subtilis pullulanase |
|---|---|
| Data collection | Spring-8 BL38B1 |
| wavelength (\acute{Å}) | 0.8 |
| detector | Jupiter 210 CCD |
| space group | P2₁2₁2₁ |
| lattice constant (\acute{Å}) | A = 70.57, b = 127.68, c = 189.25 |
| resolution (\acute{Å}) | 46.3-2.1 (2.18-2.10) |
| measured reflection | 590,744 (38,618) |
| unique reflection | 99,003 (9,775) |
| integrity (%) | 99.6 (99.6) |
| Rmerge (%) | 7.8 (31.4) |
| determination of structure | molecule substitution method |
| Refinement | |
| Residues/Water/Ca2+/α-CD | 712 × 2, 673, 2, 0 |
| Resolution (\acute{Å}) | 15.0-2.1 (2.17-2.1) |
| used reflection | 98,636 (8,725) |
| r.m.s. bond (\acute{Å}), angle (°) | 0.005, 1.25 |
| R and Rfree | 0.201, 0.238 (0.224, 0.252) |

3. X-ray Analysis of Bacillus subtilis Pullulanase (in a State of Containing α-cyclodextrin (CD))

Since α-cyclodextrin (CD) is cyclic oligosaccharide and is an analog having a structure of amylose helix consisting of six glucose residues, it has often been used for studying as an inhibitor of an amylolytic enzyme such as amylase and pullulanase. In order to determine the substrate binding site of Bacillus subtilis pullulanase, an analysis of the three-dimensional structure of enzyme containing CD as a ligand (hereinafter, referred to as “CD-containing Bacillus subtilis pullulanase”) was carried out. The analysis of the three-dimensional structure was carried out basically by the same method as 2. except that purified Bacillus subtilis recombinant pullulanase was prepared in 1. and 20 mM CD was added in the hanging drop at the time of crystallization. Data related to statistics about data collection and data are shown in Table 2.

TABLE 2

| | Bacillus subtilis pullulanase containing |
|---|---|
| Crystal | α-cyclodextrin |
| Data collection | Spring-8 BL38B1 |
| wavelength (\acute{Å}) | 1.0 |
| detector | Jupiter 210 CCD |
| space group | P2₁2₁2₁ |
| lattice constant (\acute{Å}) | A = 70.36, b = 127.86, c = 189.29 |
| resolution (\acute{Å}) | 40.0-2.2 (2.28-2.20) |
| measured reflection | 4.08, 687 (35,910) |
| unique reflection | 87,392 (8,550) |
| integrity (%) | 99.8 (99.3) |
| Rmerge (%) | 9.2 (42.4) |
| determination of structure | molecule substitution method |
| Refinement | |
| Residues/Water/Ca2+/α-CD | 712 × 2, 673, 2, 2 |
| Resolution (\acute{Å}) | 15.0-2.2 (2.28-2.2) |
| used reflection | 87,004 (8,495) |
| r.m.s. bond (\acute{Å}), angle (°) | 0.005, 1.25 |
| R and Rfree | 0.197, 0.238 (0.249, 0.282) |

A model of the three-dimensional structure of the obtained CD-containing Bacillus subtilis pullulanase is shown in FIG. 1. Note here that data of atomic coordinate are shown in the last part of the specification.

4. Comparison Analysis Between Three-dimensional Structure of CD-containing Bacillus subtilis Pullulanase and Three-dimensional Structure of Klebsiella pneumoniae Pullulanase

The three-dimensional structure of CD-containing Bacillus subtilis pullulanase obtained in 3. and the three-dimensional structure of Klebsiella pneumoniae pullulanase (J. Mol. Biol., 359 (3): 690-707 (2006), RCSB Protein Data Bank code 2FSZ) were superimposed onto each other by Least Square method by using program TURBO-FRODO, α carbons of amino acids of 499 residues were common within 2 Å with the root means square deviation (r.m.s.d.) of 1.09 Å. FIG. 2 shows the superimposed results. FIG. 3 is an enlarged view showing a CD binding site. FIG. 3 shows a CD binding site with respect to Bacillus subtilis pullulanase and the binding site of maltotetraose with respect to Klebsiella pneumoniae pullulanase (G4) (see, non-patent document 1). An amino acid residue (upper stage) in a position capable of forming CD and hydrogen binding in Bacillus subtilis pullulanase is shown together with the corresponding amino acid residue (lower stage) in Klebsiella pneumoniae pullulanase. Furthermore, amino acid residue (lower stage) that has been deduced to be involved in binding to G4 in Klebsiella pneumoniae pullulanase and the corresponding amino acid residue (upper stage) in Bacillus subtilis pullulanase are also shown together.

Although a domain (N1 domain) in the N terminal region observed in Klebsiella pneumoniae pullulanase was not observed in Bacillus subtilis pullulanase, both enzymes were very similar to each other in a catalytic region and a domain (A domain) as a nucleus including a substrate binding region. Furthermore, a CD binding site of Bacillus subtilis pullulanase is in the vicinity of G4 binding site of Klebsiella pneumonia.

As shown in FIG. 3, in Bacillus subtilis pullulanase, twelve in total of amino acid residues that are thought to be involved in binding to the substrate were successfully identified as an amino acid position in the binding site of CD: Y292 (tyrosine the 292 position), G371 (glycine at the 371 position), D406 (aspartic acid at the 406 position), L407 (leucine at the 407 position), W437 (tryptophane at the 437 position), D465 (aspartic acid at the 465 position), T475 (threonine at the 475 position), F476 (phenyl alanine at the 476 position), D525 (aspartic acid at the 525 position), N526 (asparagine at the 526 position), N580 (asparagine at the 580 position), and Y582 (tyrosine at the 582 position). Furthermore, in Klebsiella pneumoniae pullulanase, amino acid corresponding to these amino acid residues, that is, Y559 (tyrosine at the 582 position), C643 (cysteine at the 643 position), D677 (aspartic acid at the 677 position), L678 (leucine at the 678 position), W708 (tryptophane at the 708 position), D734 (aspartic acid at the 734 position), P745 (proline at the 745 position), F746 (phenyl alanine at the 746 position), D834 (aspartic acid at the 834 position), N835 (asparagine at the 835 position), D890 (aspartic acid at the 890 position), and Y892 (tyrosine at the 892 position) were successfully determined. Among the thus determined amino acids, D677, W708, D734, D834, N835 and D890 were amino acids deduced to be involved in binding to G4 in the above-mentioned report.

5. Comparison Analysis Between Three-dimensional Structure of CD-containing Bacillus subtilis Pullulanase and Three-dimensional Structure of Pseudomonas amyloderamosa Isoamylase

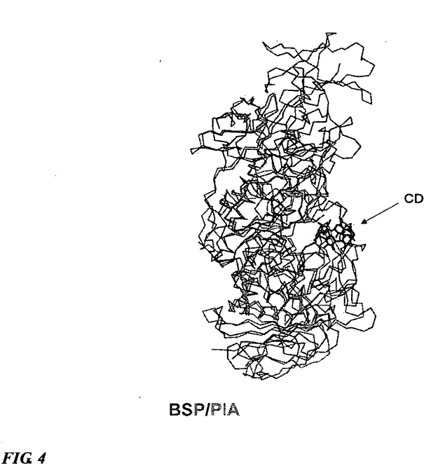

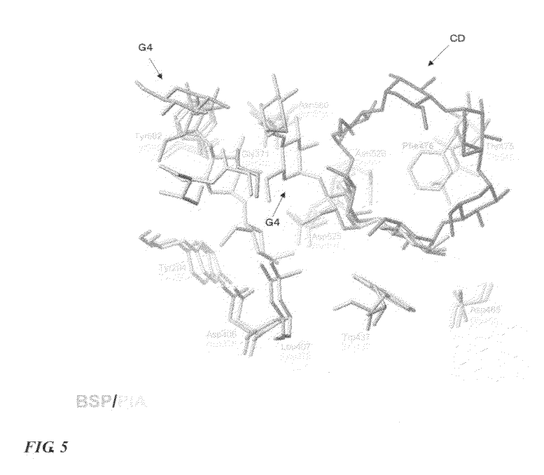

The three-dimensional structure of CD-containing Bacillus subtilis pullulanase obtained in 3. and the three-dimensional structure of Pseudomonas amyloderamosa isoamylase (J. Mol Biol., 281. (5): 885-97 (1998), RCSB Protein Data Bank code IBF2) were superimposed onto each other by Least Square method by using program TURBO-FRODO, α carbons of amino acids of 421 residues were common within 2 Å with the root means square deviation (r.m.s.d.) of 1.13 Å. FIG. 4 shows the superimposed results. FIG. 5 is an enlarged view showing a CD binding site.

The both enzymes do not have a domain (N1 domain) in the N terminal region observed in Klebsiella pneumoniae pullulanase and is very similar in domain (A domain) in terms of, for example, a nucleus including a catalytic region or a substrate binding region. The amino acid residue (upper stage) in the position capable of forming hydrogen binding to CD in Bacillus subtilis pullulanase is shown together with the corresponding amino acid residue (lower stage) in Pseudomonas amyloderamosa isoamylase. Furthermore, amino acid residues corresponding to the amino acids deduced to be involved in binding to G4 in Klebsiella pneumoniae pullulanase (upper stage: Bacillus subtilis pullulanase, and lower stage: Pseudomonas amyloderamosa).

### 6. Formation of Alignment of Amino Acid Sequence Based on Three-dimensional Structure

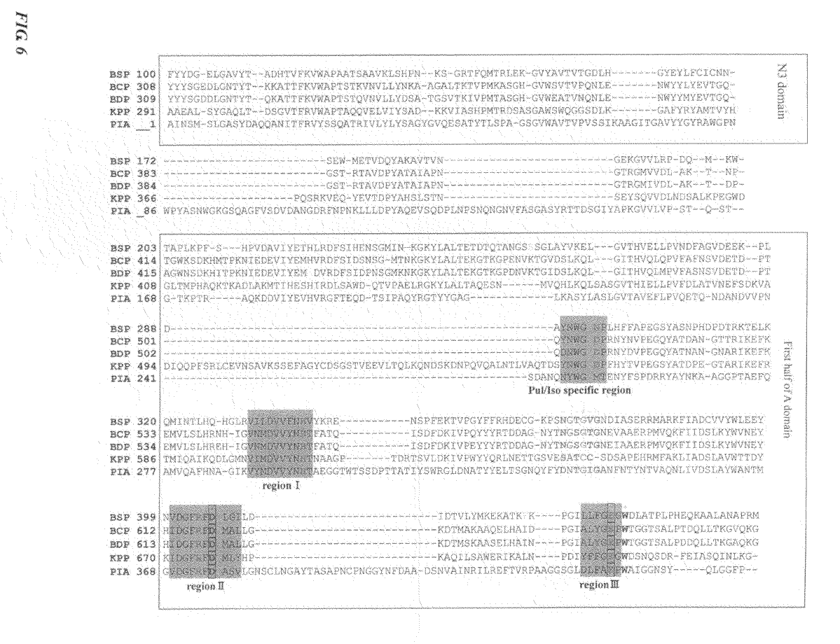

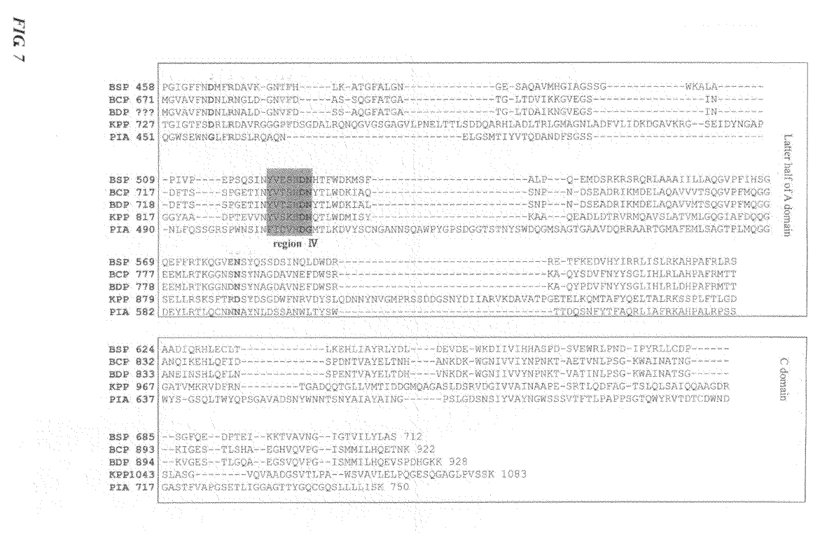

With the use of the result of comparison of the three-dimensional structures of CD-containing Bacillus subtilis pullulanase, Klebsiella pneumoniae pullulanase and Pseudomonas amyloderamosa isoamylase obtained in 4. and 5., alignments of amino acid sequences with respect to these three enzymes were formed based on the three-dimensional structures. Furthermore, alignments of amino acid sequences with respect to two kinds of pullulanases (pullulanase from Bacillus sp. APC-9603 and pullulanase from Bacillus deramificans) being derived from strains related to Bacillus subtilis and having a high homology in primary structure were formed by using ClustalW program. Alignments were formed with respect to five kinds of enzymes with these two alignments added (see FIGS. 6 and 7). As shown in FIGS. 6 and 7, these five kinds of enzymes have extremely high partial similarity.

7. Production of Bacillus subtilis Recombinant Pullulanase Mutant

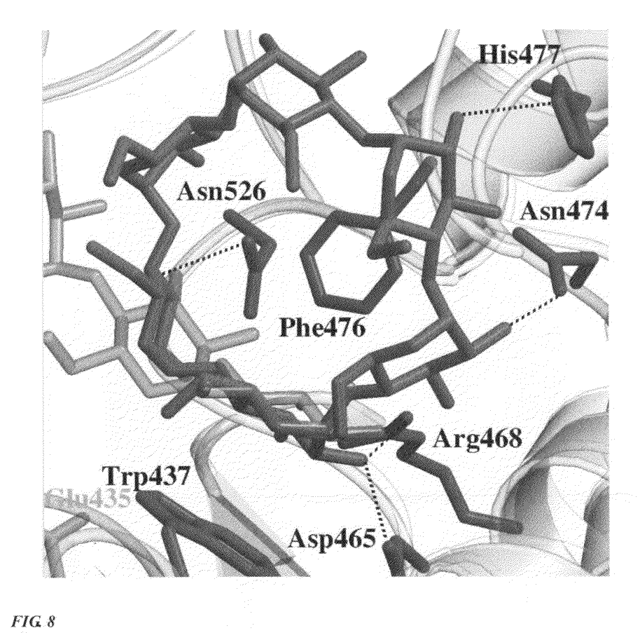

From the three-dimensional structure of Bacillus subtilis pullulanase to which CD obtained in 3. was bonded as a ligand, it is assumed that the side chain of Phe476 of Bacillus subtilis pullulanase is incorporated in the cyclic structure of the CD as a substrate analog (FIG. 8) and has something to do with the recognition of a substrate. Under this assumption, Phe476 was substituted with another amino acid by the following procedure and the effect thereof was verified.

Based on the sequence of gene AmyX encoding Bacillus subtilis pullulanase, a primer for substituting Phe476 with Gly or Ser was synthesized. In order to substitute Phe476 with Gly or Ser, forward primers and the corresponding reverse primers set forth in SEQ ID NOs: 5 and 6 were synthesized, respectively, and the concentration was adjusted to 100 ng/μl. By using an expression plasmid pEBSP of Bacillus subtilis recombinant pullulanase as a template, the following PCR reaction was carried out: 1.5 ill (30ng/μl) of pEBSP, 5 μl of 10×PCR buffer solution (one attached to the below-mentioned DNA polymerase), 1.0 μl (2.5 mM each) of dNTP, 1.25 μl each of mutated primer sets (forward and reverse primers), 39 μl of sterile water and 1 μl (2.5 U) of Pfu Turbo Hotstart DNA polymerase (Stratagene) were prepared; 30 cycles of reactions at 95° C. for 30 seconds (denaturation)—at 55° C. for 60 seconds (annealing)—at 68° C. for 12 minutes (elongation) were carried out; and finally amplification at 68° C. for 5 minutes was carried out. The obtained PCR product was confirmed by 1% agarose gel electrophoresis, and then the rest of the PCR product was treated with restriction enzyme Dpn I to degrade a methylated template plasmid and transformed to Escherichia coli competent cell DH5α strain. Plasmid DNA was isolated from the obtained ampicillin resistance transformant, the base sequence was confirmed and the intended mutated gene was obtained. A plasmid having the intended mutated gene was introduced into an expression host Escherichia coli HMS174 (DE3) strain to express and purify the mutated pullulanase of Bacillus subtilis similar to 1. (2) and (3).

SEQ ID NO: 5

5′-GTAAAAGGGAACACCGGTCACCTTAAGGCAATA-3′

SEQ ID NO: 6

5′-GTAAAAGGGAACACCTCTCACCTTAAGGCAATA-3′

8. Substrate Specificity of Bacillus subtilis Recombinant Pullulanase Mutant

The kinetic parameters with respect to pullulan and amylopectin of Bacillus subtilis recombinant pullulanase mutant prepared in 4. were measure according to the method described in J Biochem (Tokyo), 116(6): 1264-8 (1994). Pullulan or amylopectin having various concentrations were dissolved in 50 mM acetate buffer (pH 5.6) and reacted at 25° C. The concentration of a reducing sugar contained in the regularly sampled reaction solution was determined by a Park-Johnson method, and the reaction rate was measured from the increasing rate of the reducing sugar. Km value and Kcat value were obtained by curve fitting into Michaelis-Menten equation by the non-linear minimum square method. Results are shown in Table 3.

TABLE 3

| | |
|---|---|
| pullulan | amylopectin |

| | Km | kcat | Km | kcat |
|---|---|---|---|---|
| | (mg/ml) | (s⁻¹) | (mg/ml) | (s⁻¹) |
| wild type pullulanase | 14.93 | 4317 | 199.20 | 4121 |
| mutant type (F476G) | 20.45 | 5362 | 53.19 | 1198 |
| mutant type (F476S) | 645.16 | 10365 | 156.25 | 2146 |

When the wild type and the mutant types are compared with each other, they are shown to be different in the Km value and Kcat value. For example, in the mutant type (F476G: mutated enzyme in which an amino acid residue at the 476 position is changed from phenyl alanine to glycine), the Km value when amylopectin is used as a substrate is considerably lower than that of the wild type, which shows that the affinity to amylopectin is radically improved. Furthermore, in the mutant type (F476S: mutated enzyme in which an amino acid residue at the 476 position is changed from phenyl alanine to serine), the Km value when amylopectin is used as a substrate is lower than that of the wild type while the Km value when pullulan is used as a substrate is radically higher than that of the wild type, which shows that the substrate specificity has radically changed. Thus, introduction of mutation enabled the action property with respect to pullulan and amylopectin to be changed.

9. Cloning of Klebsiella pneumoniae Pullulanase Gene
(1) Cloning of Klebsiella pneumoniae Pullulanase Gene

Two primers (SEQ ID NOs: 7 and 8) were synthesized with reference to the base sequence of pullulanase of Klebsiella aerogence W70 strain (J. Bacteriol. 169 (5), 2301-2306 (1987) and J Bacteriol. 174(9): 3095.(1992), GenBank accession No. M16187). The PCR reaction was carried out under the following conditions by using chromosome DNA of Klebsiella pneumoniae ATCC9621 strain that had been isolated by the method by Sambrook et al. (Molecular Cloning: a laboratory manual, 2nd Edition, Cold Spring harbor Laboratory Press, 1989) as a template. The PCR reaction was carried out by using Accu Taq™ LA DNA polymerase system (Sigma), the reaction included one cycle of reaction at 98° C. for 30 seconds—at 59° C. for 20 seconds—at 68° C. for 3 minutes; 29 cycles of reaction at 98° C. for 15 seconds—at 59° C. for 20 seconds—at 68° C. for 3 minutes; and reaction at 68° C. for 10 minutes of amplification was carried out. The obtained PCR fragment was subjected to TA cloning by using a pGEM-T vector (Promega). It was confirmed that the obtained PCR fragment encodes pullulanase correctly by determining the base sequence. The base sequence of the obtained PCR fragment and the amino acid sequence encoded thereby are shown in SEQ ID NO: 12 and SEQ ID NO: 13, respectively.

SEQ ID NO: 7

5′-TTATTGCCGGAGAGTGGCGA-3′

SEQ ID NO: 8

5′-CCAGACTGCTGACAAAGTGC-3′

(2) Expression of Klebsiella pneumoniae Pullulanase Gene

In order to construct an expression vector, primers shown in SEQ ID NOs: 9 and 10 were synthesized, and PCR reaction was carried out by using a pullulanase gene of Klebsiella pneumoniae as a template. The obtained PCR product was treated with restriction enzymes Nde I and Xba I, and linked to a plasmid pCold-I (TAKARA) to obtain an expression plasmid pCold-KPP of Klebsiella pneumoniae pullulanase. The plasmid was introduced into Escherichia coli JM109 (TAKARA) by transformation. Culture of the obtained transformant and purification of the expressed recombinant pullulanase were carried out according to the method described in 1.

SEQ ID NO: 9

5′-GGAATTCCATATGGATGTCGTCGTCCGCTTACCG-3′ (34 mer)

SEQ ID NO: 10

5′-GCTCTAGATTATTTACTGCTCACCGGCAG-3′ (29 mer)

10. Production of Klebsiella pneumoniae Pullulanase Mutant

The results obtained by analysis by superimposing the three-dimensional structure of CD-containing Bacillus subtilis pullulanase obtained in 3. and the three-dimensional structure of Klebsiella pneumoniae pullulanase (RCSB Protein Data Bank code 2FSZ) onto each other (FIGS. 2 and 3) and the results of amino acid sequence alignments (FIGS. 6 and 7), an amino acid residue of pullulanase of Klebsiella pneumonia corresponding to Phe 476 residue that was thought to be involved in recognition of a substrate in Bacillus subtilis pullulanase was thought to be a Phe746 residue. Then, the Phe746 residue of pullulanase of Klebsiella pneumonia was substituted with Ala (or other amino acid).

Forward primers and the corresponding reverse primers set forth in SEQ ID NO: 11 were synthesized respectively in order to replace Phe746 with Ala in Klebsiella pneumoniae, and the concentration was adjusted to 100 ng/μl. By using an expression plasmid pCold-KPP of Klebsiella pneumoniae pullulanase as a template, the following PCR reaction was carried out: 1.5 μl (30 ng/pl) of pEBSP, 5 μl of 10×PCR buffer solution (one attached to the below-mentioned DNA polymerase), 1.0 μl (2.5 mM each) of dNTP, 1.25 μl each of mutated primer sets (forward and reverse primers), 39 μl of sterile water and 1 μl (2.5 U)) of Pfu Turbo Hotstart DNA polymerase (Stratagene) were prepared; 30 cycles of reactions at 95° C. for 30 seconds (denaturation)—at 55° C. for 60 seconds (annealing)—at 68° C. for 12 minutes (elongation) were carried out; and finally amplification at 68° C. for 5 minutes was carried out. The obtained PCR product was confirmed by 1% agarose gel electrophoresis, and then the rest of the PCR product was treated with restriction enzyme Dpn I to degrade a methylated template plasmid and transformed to Escherichia coli competent cell DH5α strain. Plasmid DNA was isolated from the obtained ampicillin resistance transformant, the base sequence was confirmed and the intended mutated gene was obtained. A plasmid having the intended mutated gene was introduced into an expression host Escherichia coli HMS174 (DE3) strain to express and purify the mutated pullulanase of Klebsiella pneumoniae similar to 1. (2) and (3).

SEQ ID NO: 11

5′-GCCGGCCGACTCCGGTGAC-3′

11. Substrate Specificity of Klebsiella pneumoniae Pullulanase Mutant

The kinetic parameters with respect to pullulan and amylopectin of mutated pullulanase (F746A) and wild type pullulanase (WT) of Klebsiella pneumoniae prepared in 10. were measure according to the method described in J Biochem (Tokyo), 116(6): 1264-8 (1994). Pullulan or amylopectin having various concentrations were dissolved in 50 mM acetate buffer (pH 5.6) and reacted at 25° C. The concentration of a reducing sugar contained in the regularly sampled reaction solution was determined by a Park-Johnson method, and the reaction rate was measured from the increasing rate of the reducing sugar. Km value and Kcat value were obtained by curve fitting into Michaelis-Menten equation by the non-linear minimum square method. Results are shown in Table 4.

TABLE 4

| | pullulan | | amylopectin | |
|---|---|---|---|---|
| | Km (%) | Kcat (s⁻¹) | Km (%) | Kcat (s⁻¹) |
| wild type pullulanase | 0.00091 | 52.0 | 0.0063 | 7.0 |
| mutant type (F746A) | 0.00045 | 18.2 | 0.0083 | 5.2 |

Km value when pullulan was used for a substrate was lowered in the case of mutated enzyme as compared with the case of the wild type enzyme. On the other hand, Km value when amylopectin was used for a substrate was increased. From the results, it can be evaluated that in the mutated enzyme, the affinity to pullulan is improved (the affinity to amylopectin is lowered). Furthermore, Kcat value is different between the wild type and the mutant type. Thus, introduction of mutation enabled the action property with respect to pullulan and amylopectin to be changed.

Note here that the enzyme in which the affinity to a certain substrate is improved can act on a smaller amount of substrate. Such a property is advantageous in that the presence of the small amount of substrates is required to be measured.

### INDUSTRIAL APPLICABILITY

A designing method and a preparation method of the present invention are used for improving an enzyme hydrolyzing an α-1,6-glycosidic linkage. With a mutated enzyme whose affinity and specificity to a substrate are improved, the increase in the yield of products and reduction of the amount of enzyme to be used (additive amount) can be achieved. On the other hand, it can be expected that the use of the designing method and the preparation method of the present invention provide a mutated enzyme usable to a novel application that has not been assumed with the use of wild type enzymes. That is to say, the present invention is capable of contributing to expansion of the applications of use of enzymes hydrolyzing an α-1,6-glycosidic linkage.

The present invention is not limited to the description of the above exemplary embodiments and Examples. A variety of modifications, which are within the scopes of the following claims and which are easily achieved by a person skilled in the art, are included in the present invention.

Contents of the theses, Publication of Patent Applications, Patent Publications, and other published documents referred to in this specification are herein incorporated by reference in its entity.

The atomic coordinates of three-dimensional structure of CD-containing Bacillus subtilis (Bacillus subtilis strain 168) are shown below.

Lengthy table referenced here
US20090280553A1-20091112-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20090280553A1). An electronic copy of the table will also be available from the USPTO upon request and payment of the fee set forth in 37 CFR 1.19(b)(3).

1. A method for designing a mutated enzyme, the method comprising following steps:
(1) specifying one or two or more amino acids selected from the group shown below in an amino acid sequence of an enzyme (enzyme to be mutated) that hydrolyzes an α-1,6-glycosidic linkage, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2; and
(2) constructing an amino acid sequence in which the amino acid specified in the step (1) is substituted with another amino acid or deleted based on the amino acid sequence of the enzyme to be mutated.

2. The method for designing a mutated enzyme according to claim 1, wherein in the step (1), one or two or more amino acids selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position, and an amino acid corresponding to an amino acid at the 582 position of the amino acid sequence set forth in SEQ ID NO: 2 is specified.

3. The method for designing a mutated enzyme according to claim 1, wherein in the step (1), an amino acid corresponding to an amino acid at the 476 position of an amino acid sequence set forth in SEQ ID NO: 2 is specified.

4. The method for designing a mutated enzyme according to claim 1, wherein the specifying of an amino acid in step (I) is carried out by comparing between an amino acid sequence of the enzyme to be mutated and the amino acid sequence set forth in SEQ ID NO: 2 and/or between a three-dimensional structure of the enzyme to be mutated and a three-dimensional structure of the amino acid sequence set forth in SEQ ID NO: 2.

5. The method for designing a mutated enzyme according to claim 1, wherein the enzyme to be mutated is a wild type enzyme.

6. The method for designing a mutated enzyme according to claim 1, wherein the enzyme to be mutated is pullulanase or isoamylase derived from a microorganism.

7. The method for designing a mutated enzyme according to claim 6, wherein the microorganism is a microorganism of genus bacillus, a microorganism of genus Klebsiella, or a microorganism of genus pseudomonas.

8. The method for designing a mutated enzyme according to claim 1, wherein the amino acid sequence of the enzyme to be mutated is an amino acid sequence having a 70% or more identity to the amino acid sequence set forth in SEQ ID NO: 2.

9. The method for designing a mutated enzyme according to claim 1, wherein the amino acid sequence of the enzyme to be mutated is an amino acid sequence set forth in any of SEQ ID NOs:2, 13 to 16.

10. A method for preparing a mutated enzyme, the method comprising following steps:
(1) preparing a nucleic acid encoding an amino acid sequence constructed by the method described in an claim 1;
(2) expressing the nucleic acid; and
(3) collecting expression products.

11. A mutated enzyme comprising an amino acid sequence in which one or two or more amino acids selected from the group shown below in an amino acid sequence of an enzyme (enzyme to be mutated) that hydrolyzes an α-1,6-glycosidic linkage, the group consisting of an amino acid corresponding to an amino acid at the 292 position, an amino acid corresponding to an amino acid at the 371 position, an amino acid corresponding to an amino acid at the 406 position, an amino acid corresponding to an amino acid at the 407 position, an amino acid corresponding to an amino acid at the 437 position, an amino acid corresponding to an amino acid at the 465 position, an amino acid corresponding to an amino acid at the 475 position, an amino acid corresponding to an amino acid at the 476 position; an amino acid corresponding to an amino acid at the 525 position, an amino acid corresponding to an amino acid at the 526 position, an amino acid corresponding to an amino acid at the 580 position and an amino acid corresponding to an amino acid at the 582 position of the amino acid sequence set forth in SEQ ID NO: 2 is substituted with another amino acid or deleted.

12. The mutated enzyme according to claim 11, wherein the substituted or deleted amino acid is one or two or more amino acids selected from the group consisting of an amino acid corresponding to an amino acid at the 292 position, an amino acid corresponding to an amino acid at the 371 position, an amino acid corresponding to an amino acid at the 407 position, an amino acid corresponding to an amino acid at the 475 position, an amino acid corresponding to an amino acid at the 476 position, and an amino acid corresponding to an amino acid at the 582 position of the amino acid sequence set forth in SEQ ID NO: 2.

13. The mutated enzyme according to claim 11, wherein the substituted or deleted amino acid is an amino acid corresponding to an amino acid at the 476 position of the amino acid sequence set forth in SEQ ID NO: 2.

14. The mutated enzyme according to claim 11, wherein the enzyme to be mutated is a wild type enzyme.

15. The mutated enzyme according to claim 11, wherein the enzyme to be mutated is pullulanase or isoamylase derived from a microorganism.

16. The mutated enzyme according to claims 15, wherein the microorganism is a microorganism of genus bacillus, a microorganism of genus Klebsiella, or a microorganism of genus pseudomonas.

17. The mutated enzyme according to claim 11, wherein the amino acid sequence of the enzyme to be mutated is an amino acid sequence having a 70% or more homology with respect to the amino acid sequence set forth in SEQ ID NO: 2.

18. The mutated enzyme according to claim 11, wherein the amino acid sequence of the enzyme to be mutated is an amino acid sequence set forth in any of SEQ ID NOs: 2, 13 to 16.

19. The mutated enzyme according to claim 11, wherein an action property with respect to pullulan or an action property with respect to amylopectin is improved as compared with the enzyme to be mutated.

20. A gene encoding the mutated enzyme according to claim 11.

21. A recombinant DNA including the gene according to claim 20.

22. A microorganism carrying the recombinant DNA according to 21.
